SEQUENCE LISTING (80 sequences)

SEQ ID NO: 1 - RNA, 128 nt, Setaria italica
cggugagagcucucugcucugguagccaaggaugacuugccuguguuggccuccggcugcaggaggcucuccugcggcguguggucucgcaggcagucuccuuggcuagccugagcggcucucauccu

SEQ ID NO: 2 - RNA, 153 nt, Setaria italica
cgauaggagccuguccagauagccaaggaugacuugccuguguggccucuuggagugagcucgagcuuagcuagccuugugcaugaugaugcuuugcucucuucccguggucucacaggcagucuccuuggcuaguccgggcggcucuuaucu

SEQ ID NO: 3 - RNA, 147 nt, Setaria italica
gcgaugagggcucugcuacgguagccaaggaugacuugccuguggucuccugcugcaggaggcaacccauuuggcguggaucugcucucugcugcgcgcgguuggucucgcaggcagucuccuuggcuauccugaggggcucccauc

SEQ ID NO: 4 - RNA, 157 nt, Oryza sativa
ggucuugcauggaaguaagaggccaucuucgauagccaaggaugauuugccuguagccccauugucaucagcucucuccgucgagagcgacaaccgggcucuacuggcaagucuccucggcuacccgaguaccucuuaugcuaucccaugucagacc

SEQ ID NO: 5 - RNA, 150 nt, Sorghum bicolor
gcgauggaagcucugcuuugguagccaaggaugagcugccuguggccuccagcugcagaggcuagcuaggcuacacauugcguggccaagcuccuccgcugcgcguggucucgcaggcagccuccuuggcuagucugaguggcuuccauc

SEQ ID NO: 6 - RNA, 133 nt, Zea mays
gcgauggaagcucugcuucgguagccaaggaugagcugccuguggccuccugcugcggacguugcguggccccgccuccaccgcgugcgguccccgcaggcagccuccuuggcuagucugagcggcuuccauc

SEQ ID NO: 7 - RNA, 101 nt, Setaria italica
auagccaaggaugauuugccuguagccaccucugaaugcuccuggugccauggcaaucaggagcagcaaguggcggcccuccgggcaaaucauccgggcua

SEQ ID NO: 8 - RNA, 201 nt, Setaria italica
agagagcaaggcuuacauggcgaugaggguuuuaguagcucugguagccaaggaugacuugccugugucgaccugaucuuucccugaaaggaucgucgucgucaucagcagauggucgucgaugagccuugggguggucucaccggcagucuccuuggcuagccuggcucacucuccucgcucaugcuagcauugcaucuc

SEQ ID NO: 9 - RNA, 166 nt, Setaria italica
agauagcaagccuuacauggcgauaagaguuuggcucugguagccaaggaugacuugccuguguccucucuggaggaucaacaaauauugaggcuuugaauggucucaugggcagucuccuuggcuagccugaguggcucuuauugcucaugcuagacuugcaucu

SEQ ID NO: 10 - RNA, 102 nt, Setaria italica
cugguagccaaggaugacuugccuguggccucccucgcucgcuugcauuuugguggagcaggcaggcaacugaucucauaggcagucuccuuggcuagccug

SEQ ID NO: 11 - RNA, 135 nt, Setaria italica
gcaacaggagccgcucagacuagccuaggagacugccuaugagccaucucaaaggcucacacacugcugacugcugauccuucaguauaaaggaccuaggcaagucauccuuggcuaucagaggcaggcccuuau

SEQ ID NO: 12 - RNA, 135 nt, Setaria italica
auaagggccugccucugauagccaaggaugacuugccuagguccuuuauacugaaggaucagcagucagcagugugugagccuuugagauggcucauaggcagucuccuaggcuagucugagcggcuccuguugc

SEQ ID NO: 13 - RNA, 128 nt, Glycine max
aagagacagucaaccagccaaggaugccugccaauaaaccaaucuaagggcuccgucuucauuagagacaaaguccuacaaaugggaauagcuaggcaagucauccuuggcuauccgacaugacucuu

SEQ ID NO: 14 - RNA, 135 nt, Glycine max
aagagucaugucugguagccaaggaugacuugccuaguuauuccauuuuaugggacuuuauuuaauuaauuugucucauauggagacccuuaguuugguuaauuggcaagcuuccucggcuauuugcaugccucu

SEQ ID NO: 15 - RNA, 136 nt, Glycine max
aagagucaugucugauagccaaggaugacuugccuagcuauuccauuuuaugggacuuuauuuaauuaauuugucucauauggagacccuuaguuugguuaauuggcaagcuuccucggcuauuugcaugccucuu

SEQ ID NO: 16 - RNA, 162 nt, Manihot esculenta
ugauagccaaggaugacuugccuaucuccuccaacaggguuucaaacagaugaauaaugcagaagaaugcggugggauuaaucuggauuuggagcucuuuuguggcucauaggcagucuccuucggcuagucugacuggcucuuaucuuucaugcuaggccu

SEQ ID NO: 17 - RNA, 200 nt, Manihot esculenta
aggagaggaagagaggccuuguaugaagaugaagagcuacguaugauagccaaggaugacuugccuaucuccugccguagauauauaaaaaaaaaugcacaagaaagaauauggugggagguucuuucguauggguggagcuaggcagucuccuuuggcuagucucacuggcucucaucuuucaugcuaggccugcaucu

SEQ ID NO: 18 - RNA, 115 nt, Vitis vinifera
cuugugugguagccaaggaugacuugccugcuccucccgagugguucguggggauauagauuauagaccccugugugguccucaggcagucaccuuggcuaacuugacaggcacu

SEQ ID NO: 19 - DNA, 111 bp, Sorghum bicolor
tagccaaggatgatttgcctgtagctagcaacctctgagcgctcctgctgccatggcatggcagtcaggggcgcgtagtgggtgcttctccgggcaaatcatctgggctag

SEQ ID NO: 20 - DNA, 142 bp, Sorghum bicolor
aagaggcatctttgatagccagggatgatttgccctgtagcaccatgcatgcatgcaacctctcgcgttagctcctgctgactgcatgctgccatgacaagttccacgggcaaatcattcctggctaatctgagtgcctctt

SEQ ID NO: 21 - DNA, 149 bp, Setaria italica
gcgatggaagctctgctttggtagccaaggatgagctgcctgtggcctccagctgcagaggctagctaggctacacattgcgtggccaagctcctccgctgcgcgtggtctcgcaggcagcctccttgctagtctgagtggcttccatc

SEQ ID NO: 22 - DNA, 132 bp, Zea mays
gcgatggaagctctgcttcggtagccaaggatgagctgcctgtggcctcctgctgcggacgttgcgtggccccgcctccaccgcgtgcggtccccgcaggcagcctccttggctagtctgagcggctccatc

SEQ ID NO: 23 - RNA, 126 nt, Sorghum bicolor
gcaauaggggccacucaggcuagccaaggagacugccuaugaaccaacucaaagguucacauucugauccuuugggacaaaggacauaggcaagucauccuuggcuaucagaggcagacccuuauu

SEQ ID NO: 24 - RNA, 126 nt, Sorghum bicolor
aauaagggucugccucugauagccaaggaugacuugccuauguccuuugucccaaaggaucagaaugugaaccuuugaguugguucauaggcagucuccuuggcuagccugaguggccccuauugc

SEQ ID NO: 25 - DNA, 21 bp, Sorghum bicolor
taggcaagtcatccttggcta

SEQ ID NO: 26 - DNA, 21 bp, Sorghum bicolor
tagccaaggatgacttgccta

SEQ ID NO: 27 - RNA, 20 nt, Sorghum bicolor
ucgguuccuacugaacggau

SEQ ID NO: 28 - RNA, 21 nt, Sorghum bicolor
aucgguuccuacugaacggau

SEQ ID NO: 29 - RNA, 19 nt, Sorghum bicolor
aucgguuccuacugaacgg

SEQ ID NO: 30 - RNA, 18 nt, Sorghum bicolor
ucgguuccuacugaacgg

SEQ ID NO: 31 - RNA, 20 nt, Sorghum bicolor
agccaagaaugauuugccua

SEQ ID NO: 32 - RNA, 20 nt, Sorghum bicolor
agccaagaaugaauugccug

SEQ ID NO: 33 - RNA, 20 nt, Sorghum bicolor
agccaagggauacuuguuua

SEQ ID NO: 34 - RNA, 21 nt, Sorghum bicolor
ugcccaagcauggcuugccug

SEQ ID NO: 35 - RNA, 19 nt, Sorghum bicolor
ugcccaagcauggcuugcc

SEQ ID NO: 36 - RNA, 21 nt, Sorghum bicolor
uugauaaggauggcuugccug

SEQ ID NO: 37 - RNA, 20 nt, Sorghum bicolor
agccaaggagaacuugucuu

SEQ ID NO: 38 - RNA, 18 nt, Sorghum bicolor
ggccaaugaugauuugcc

SEQ ID NO: 39 - RNA, 18 nt, Sorghum bicolor
agcaaaggaugauuugca

SEQ ID NO: 40 - RNA, 18 nt, Sorghum bicolor
agccgaugaugauuugcu

SEQ ID NO: 41 - RNA, 18 nt, Sorghum bicolor
cgccaaagaugacuugcu

SEQ ID NO: 42 - RNA, 18 nt, Sorghum bicolor
ugccaaugaugacuugca

SEQ ID NO: 43 - RNA, 19 nt, Sorghum bicolor
uagccaaggaagauuuggc

SEQ ID NO: 44 - RNA, 19 nt, Sorghum bicolor
ugccggaggaugacuugcc

SEQ ID NO: 45 - RNA, 19 nt, Sorghum bicolor
uggccaaggaugauuucuc

SEQ ID NO: 46 - RNA, 19 nt, Sorghum bicolor
uagcgagggauggcuuccc

SEQ ID NO: 47 - RNA, 18 nt, Sorghum bicolor
agccaagaaugauuugcc

SEQ ID NO: 48 - RNA, 18 nt, Sorghum bicolor
guccaaggaugacuuacc

SEQ ID NO: 49 - RNA, 20 nt, Sorghum bicolor
uccguucaguaggaaccgau

SEQ ID NO: 50 - RNA, 21 nt, Sorghum bicolor
auccguucaguaggaaccgau

SEQ ID NO: 51 - RNA, 20 nt, Sorghum bicolor
uggcaacucauccuuggcuu

SEQ ID NO: 52 - RNA, 20 nt, Sorghum bicolor
aggcagcuuguacuuggcua

SEQ ID NO: 53 - RNA, 21 nt, Sorghum bicolor
uaaucaaaucauucuuggcug

SEQ ID NO: 54 - RNA, 21 nt, Sorghum bicolor
uaggcaaaucauucuuggcug

SEQ ID NO: 55 - RNA, 20 nt, Sorghum bicolor
aggcaauucauucuuggcuu

SEQ ID NO: 56 - protein, 72 aa, Arabidopsis thaliana
EEPPTDYIHVRARRGQATDSHSLAERVRREKISERMRTLQNLVPGCDKVTGKALMLDEIINYVQTLQTQVEF

SEQ ID NO: 57 - protein, 72 aa, Manihot esculenta
EEPSTGYIHVRARRGQATDSHSLAERVRREKISERMKILQQLVPGCDKVTGKALMLDEIINYVQSLQNQVEF

SEQ ID NO: 58 - protein, 72 aa, Manihot esculenta
EEPPTGYIHVRARRGQATDSHSLAERVRREKISERMKILQRLVPGCDKVTGKALMLDEIINYVQSLQNQVEF

SEQ ID NO: 59 - protein, 72 aa, Glycine max
EEPPTGYIHVRARRGQATDSHSLAERVRREKISERMKVLQRLVPGCDKVTGKALMLDEIINYVQSLQNQVEF

SEQ ID NO: 60 - protein, 66 aa, Glycine max
EDPPTGYIHVRARRGQATDSHSLAERVRREKISERMKTLQRLVTGKALVLDEIINYVQSLQNQVEF

SEQ ID NO: 61 - protein, 72 aa, Vitis vinifera
EEPPTGYIHVRARRGQATDSHSLAERVRREKISERMKLLQALVPGCDKVTGKALMLDEIINYVQSLQNQVEF

SEQ ID NO: 62 - protein, 72 aa, Brachypodium distachyon
EEAPKGYIHVRARRGQATDSHSLAERVRRERISERMRLLQTLVPGCDKVTGKALILDEIINYVQSLQNQVEF

SEQ ID NO: 63 - protein, 52 aa, Brachypodium distachyon
EEEPKGVRRERISERMRMLQALVPGCDKVTGKALILDEIINYVQSLQNQVEF

SEQ ID NO: 64 - protein, 72 aa, Oryza sativa
DEPPKGYIHVRARRGQATDSHSLAERVRRERISERMRMLQALVPGCDKVTGKALILDEIINYVQSLQNQVEF

SEQ ID NO: 65 - protein, 72 aa, Oryza sativa
EEAPQGFIHVRARRGQATDSHSLAERVRRERISERMRMLQALVPGCDKVTGKALILDEIINYVQSLQNQVEF

SEQ ID NO: 66 - protein, 72 aa, Setaria italica
DEAPKGYIHVRARRGQATDSHSLAERVRRERISERMRMLQALVPGCDKVTGKALILDEIINYVQSLQNQVEF

SEQ ID NO: 67 - protein, 71 aa, Setaria italica
EEEPKGYIHVRARRGQATDSHSLAERVRRERISERMRMLQALVPGCDKVTGKALILDEIINYVQSLQNQVE

SEQ ID NO: 68 - protein, 71 aa, Sorghum bicolor
EEPKGYIHVRARRGQATDSHSLAERVRRERISERMRVLQALVPGCDKVTGKALILDEIINYVQSLQNQVEF

SEQ ID NO: 69 - protein, 72 aa, Sorghum bicolor
DEATKGYIHVRARRGQATDSHSLAERVRRERISERMRMLQALVPGCDKVTGKALILDEIINYVQSLQNQVEF

SEQ ID NO: 70 - protein, 72 aa, Zea mays
DEASKGYIHVRARRGQATDSHSLAERVRRERISERMRMLQALVPGCDKVTGKAMVLDEIINYVQSLQNQVEF

SEQ ID NO: 71 - protein, 72 aa, Zea mays
EEEPKGYIHVRARRGQATDSHSLAERVRRERISERMRVLQALVPGCDKVTGKALVLDEIINYVQSLQNQVEF

SEQ ID NO: 72 - protein, 72 aa, Zea mays
DEATRGYIHVRARRGQATDSHSLAERVRRERISERMRMLQALVPGCDKVTGKALILDEIINYVQSLQNQVEF

SEQ ID NO: 73 - protein, 402 aa, Arabidopsis thaliana
MGTSTTESVVACEFCGERTAVLFCRADTAKLCLPCDQHVHSANLLSRKHVRSQICDNCSKEPVSVRCFTDNLVLCQECDWDVHGSCSSSATHERSAVEGFSGCPSVLELAAVWGIDLKGKKKEDDEDELTKNFGMGLDSWGSGSNIVQELIVPYDVSCKKQSFSFGRSKQVVFEQLELLKRGFVEGEGEIMVPEGINGGGSISQPSPTTSFTSLLMSQSLCGNGMQWNATNHSTGQNTQIWDFNLGQSRNPDEPSPVETKGSTFTFNNVTHLKNDTRTTNMNAFKESYQQEDSVHSTSTKGQETSKSNNIPAAIHSHKSSNDSCGLHCTEHIAITSNRATRLVAVTNADLEQMAQNRDNAMQRYKEKKKTRRYDKTIRYETRKARAETRLRVKGRFVKATDP

SEQ ID NO: 74 - protein, 195 aa, Vitis vinifera
MWDRGHLSYQAPQIWDFHLGRSRICKETSPEAGYDVDNSGFVIKNYSEITKGSSLTRTKALQGMYEMNCTTTHEDILSKNSHSNKALSSQGPTTAESNNIPIVGPSSESWTAEPNTNSIKSMQFKDLLIGSGTARTETTNVDMELLAQNRGHAMLRYKEKKKTRRYEKHIRYESRKARADTRKRVKGRFVKASDS

SEQ ID NO: 75 - protein, 362 aa, Vitis vinifera
MQEKGVPLLPNSPCALHPLAALSYPIISSISLTSDRKKTTKHTCTNNQFCYDTFLQLLTHAPHVPHCLIYYKRFWCSGFVQAVVRIELSQILLCFRVSKRQSPSCGKHKQVIFKQLGHVGESIGIENGGVLDVDHQALEQQTPFTSLLMLPNRATTGGVILWDNNPSDQSTQIWDFHLGHSRGYEECGLLEAEYGVNDAGFVIKSYSELMKETSFTNTKVVGEMYDINYSMTHEDITSFNNNSNNPTASQGAATSESNNLPIARPSSGSAFAKPKSFSGSKDIELTEQSILMRGESGRTAATTKVDLEQLAQNRGNAMLRYKEKKKTRRYDKHIRYESRKARADTRKRVKGRFVKATEAPDG

SEQ ID NO: 76 - protein, 490 aa, Brachypodium distachyon
MKEGGGRQQWPCDYCGEAAAALHCRADAARLCVACDRHVHAANALSRKHVRAPLCAACAARPAAAARLASGSSDPEFLCSACDDDGACEGAGAARVPVEGFSGCPAASELAASWGLDLLHPLPTDGCGGGGGIGRGEQEDEEDALFFSSLDYSMLVDPEMRDLYVPCDPPDSGGRPLKGEALCQQLAEMARRETQSHPPPPPQQQQYTPDLSPRTPRRSSAGPEKQHQQPPPLPQEPPFPYTSLLMNMMPPDNLAAGNNDRLRDDEAGQQLQWEFTAPSSVPPTQIWDFNLGRSRNHNENSALEVEFGSNNGGFMIKSYNDMLKEISSGTTKDLEDIYDSGYCAAAEDIMSTNICQLSSKNVSTASNKRKVSSCTSTIDGPTTSGNYVPTSGPLGSSSQDRGAALAREISFGEQTIVPTGADRPTTRIDSETLAQNRDSAMQRYREKRKNRRYEKHIRYESRKLRADTRKRVKGRFVKSNEALNASGNGG

SEQ ID NO: 77 - protein, 488 aa, Oryza sativa
MKDGGGGGGRGQQQQWPCDYCGEAAAALHCRADAARLCVACDRHVHAANALSRKHVRAPLCAACAARPAAARVASASAPAFLCADCDTGCGGDDGAALRVPVEGFSGCPAAAELAASWGLDLPGGCGGEEEEADDAFFSALDYSMLAVDPVLRDLYVPCDPPEVVVAGGGRRLKGEALGHQLAEMARREAETAHPHTQPHSDLSPRTPRRTSAAASGRLQEKQAPPPLPHAAATAAPLPYTSLLMMAPANCTELMENNRVGDEDENVLWESTAPSVPPTQIWDFNLGKSRDHNENSALEVGFGSNNGGFMIKSYNDMLKEISSGTTKDLEDIYDSRYFAAAEDIMSTNVCQLSSKNPSTRSNKRKASSCASTIDGPTTSTSHVPAASGALGGSSNDRGSALPKEISFCDQTVVPTGADQRPCTIKIDSETLAQNRDSAMQRYREKKKNRRYEKHIRYESRKLRADTRKRVKGRFVKSNGAPDDVSNGG

SEQ ID NO: 78 - protein, 336 aa, Setaria italica
MLGADPELRDLYVPCDPPEGAAHAGGARRLKGEALCDQLAEMARREADTSHPHQPHSDLSPRTPRRNSAASSGRLPGKMAPPAPPPHHPPAAVQEVPLPYTSLLMMASANCTELIGGHDRMADDDEQLLWDCAPPSVPPTQIWDFNLGRSRDHDEKSSIEVGFGSNHGGFMIKSYSDMLKEISSGTTKDLEDIYDSRYCSTAEDIMSSNICQVSSKNVSTGSNKRKVSPSTSTMDGPTTSGNHVPTSGPALTREISFGDQTVSPAGAERPAAMRIDSETLAQNRDSAMQRYREKRKNRRYEKHIRYESRKLRADTRKRVKGRFVKSTEALNAGNGG

SEQ ID NO: 79 - protein, 490 aa, Sorghum bicolor
MKSGGGGGGGGGGGGQQWPCDYCGEAAAALHCRADAARLCVACDRHVHAANALSRKHVRVPLCAGCAARPAAARVSPVPGADPAFLCAGCCDDAASAAVRVPVEGFSGCPSAAELAASWGLDLRRAEEGKDGAGGDIDDGDPFLSVLDYSVLGVAVDPDLRDLYVPCDPPRVPAPDAAGARPLRGQALCDQLAEMARRETDTAHAHPHSDLSPRTPRRTSAASGGRLPPGKMSPPAAMPTHHPPPAAVQEVPLPYTSLLMMASANCADLIGGADRVGDDDEQLLWDCAAPSVPPTQIWDFNLGRSRDHDEKSALEVGYGSNHGGFMIKSYSDMLKEISSGTTKDLEDIYDSRYCSTAEDIMSSNICQLSSKNVSTASNKRKLSSCASTIDGPTTSGNHVPTSGPALTREISFGDQTVSTPAAERPAVRIDSETLAQNRDSAMQRYREKKKNRRYEKHIRYESRKLRADTRKRVKGRFVKSTEALNAGYGG

SEQ ID NO: 80 - protein, 498 aa, Zea mays
MKSCGGGGADGQQCPCDYCGEAAAALHCRADAARLCVACDRHVHAANALSRKHVRAPLCAGCAARPAAARVSLGADPAFLCADCCEGCAAASAARVSVEGFSGCPSAAELAASWGLDLRRAAVAVGDDGDGGDDDDPFLSVLDYSVLGVGVADTDLRDLYVPCDPPRVPVPDAGARPLRGEALCDQLAEMARRDEADTSHAHPHSDLSPRTPRRTSAASSGRLPSGKMAPPAALPVPAHPPPAAPQEVPLPYTSLLMMASANCSDLIGGGDRVGDTDEQLLWDCAAPSVPPTQIWDFNLGRSRHHDEKSALEVGYGSNHGGFMIKSYSDMLKDISSGTTKDLEDIYDSRYCSTAEDIMSSNICQLSSKNVSTGSNKRKVRSCAASTMDGPTTSGNHNHVPASASGPGAALTREISFGDQTVSAPAAETERPAAVRIDSETLAQNRDSAMQRYREKKKNRRYEKHIRYESRKLRADTRKRVKGRFVKSTEALNAARYNG

(19) United States
(12) Patent Application Publication
(10) Pub. No.: US 2014/0230092 A1
(43) Pub. Date: Aug. 14, 2014

(54) miRNA169 Compositions and Methods for the Regulation of Carbohydrate Metabolism and Flowering in Plants

(71) Applicant: Rutgers, The State University of New Jersey, New Brunswick, NJ (US)

(72) Inventors: Joachim Messing, Somerset, NJ (US); Martin Calvino, Highland Park, NJ (US)

(73) Assignee: Rutgers, The State University of New Jersey, New Brunswick, NJ (US)

(21) Appl. No.: 14/160,520

(22) Filed: Jan. 21, 2014

Related U.S. Application Data
(60) Provisional application No. 61/754,745, filed on Jan. 21, 2013.

(51) Int. Cl. C12N 15/82

(52) U.S. Cl. CPC C12N 15/8271; C12N 15/8245; C12N 15/827
USPC 800/284; 435/468; 504/196; 800/278; 800/290; 800/298

(57) ABSTRACT

Compositions and methods for modulating flowering, plant height, sugar metabolism and stress response in plants are provided.

This application claims priority to U.S. Provisional Application No. 61/754,745 filed Jan. 21, 2013, the entire contents being incorporated herein by reference as though set forth in full.

### FIELD OF THE INVENTION

This invention relates to the fields of plant metabolism and molecular biology. More specifically, the invention provides miRNA169 compositions and methods for modulating expression of target nucleic acids encoding proteins involved in a variety of important biochemical pathways, including those controlling sugar metabolism, flowering and drought resistance.

### BACKGROUND OF THE INVENTION

Several publications and patent documents are cited throughout the specification in order to describe the state of the art to which this invention pertains. Each of these citations is incorporated herein by reference as though set forth in full.

Several mechanisms have been proposed to explain the evolutionary origin of miRNA genes. For instance, they can be derived from miniature inverted repeat transposable elements (MITEs) because the inverted repeat with a short internal sequence can be transcribed and form a hairpin structure that can be processed into small RNAs. Indeed, several miRNA genes derived from MITEs have been described in Arabidopsis and rice (Piriyapongsa and Jordan 2008). It has also been proposed that miRNA genes can originate from spontaneous mutations in hairpin-like structures in the genome, and several miRNAs in Arabidopsis appeared to have originated this way (Fenselau de Felippes, et al. 2008). The third, and probably the most accepted explanation for the origin of microRNAs is based on the inverted duplication of genes, which when transcribed would form hairpin structures capable of generating small RNAs with perfect complementarity to the parental transcripts (Allen, et al. 2004; Axtell and Bowman 2008). Over time, the accumulation of mutations erodes the extensive homology with the parental transcripts and the accuracy of small RNA processing improves, eventually leaving a single segment (the mature miRNA) that retains complementarity (Allen, et al. 2004; Axtell and Bowman 2008). This hypothesis is supported with evidence where extended complementarity between plant miRNAs and target mRNAs is more evident in less-conserved and younger loci (Fahlgren, et al. 2007).

Duplication of a newly formed miRNA eventually results in the creation of a multigene miRNA family, with evolutionary old and conserved miRNAs having more than one gene copy in the genome whereas new and thus non-conserved (or species-specific) miRNAs being usually single copy (Allen, et al. 2004; Fahlgren, et al. 2007; Ma, et al. 2010). Similar to protein-coding genes, duplication and subsequent divergence of miRNA gene copies can lead to loss of function (pseudogenes), keep current function (gene redundancy), gain a new function (neo-functionalization) or acquire a more specialized function (sub-functionalization) (Maher, et al. 2006). Consistent with this, diversification in the sequence of duplicated miRNA gene copies was accompanied by changes in spatial and temporal expression patterns (Jiang, et al. 2006; Maher, et al. 2006). MicroRNA genes that undergo events of tandem duplication result in the formation of paralogous miRNA gene copies located in close proximity to each other on the same chromosome, and thus forming miRNA clusters. Recently, Sun and colleagues analyzed miRNAs that had amplified through tandem duplication in Arabidopsis, poplar (Populus thricocarpa), rice (Oryza sativa) and sorghum (Sorghum bicolor) genomes, respectively; and found that 248 miRNAs in total belonging to 51 miRNA families arose by tandem duplication (Sun, et al. 2012). This study showed the importance of tandem duplication events as a major force in the creation of new miRNA gene copies and into the expansion of miRNA families. Interestingly, the average miRNA copy number in tandemly duplicated regions from eudicots A. thaliana and P. thricocarpa was lower (2.8 copies per tandem) than in monocots O. sativa and S. bicolor (3.4 copies per tandem), suggesting that tandem duplications might have been more common in rice and sorghum (Sun, et al. 2012). Despite this finding, there is a lack of knowledge on the evolutionary fate of miRNA gene clusters across the grass family.

### SUMMARY OF THE INVENTION

In accordance with the present invention, compositions comprising at least one miRNA provided in the figures or a vector encoding said at least one of said miRNA in a biologically compatible carrier for modulating expression of a plant target gene is provided. In a preferred embodiment, the target gene encodes a protein which regulates a biological parameter selected from the group consisting of flowering, stress or drought resistance, plant height, and sugar metabolism.

Also provided is a method for modulating a biological parameter selected from the group consisting of flowering, drought resistance, plant height and sugar metabolism in a plant or plant cell comprising contacting said plant or plant cell with an effective amount of the miRNA containing compositions (e.g., miRNA expressing vectors) of the invention. The compositions and methods described herein are effective for increasing production of biofuels from plants so treated.

### BRIEF DESCRIPTION OF THE DRAWINGS

FIG. 1. Distribution of MIR169 gene copies in the genome of Sorghum bicolor cultivar BTx623. A total of 22 MIR169 gene copies are shown, with 17 copies previously annotated by the sorghum genome-sequencing consortium (shown in black and red color) (Paterson, et al. 2009), and with 5 additional MIR169 copies described in this study for the first time (shown in green color). The evolutionary trajectory of sorghum MIR169 gene copies arranged in clusters 1, 2 and 3 are described.

FIG. 2. Syntenic alignment of rice and sorghum chromosomal segments containing MIR169 gene clusters. Sorghum MIR169 gene clusters on chr2 and chr7 together with their flanking protein coding genes were aligned with rice via orthologous gene pair. Rice and sorghum chromosomes are represented as horizontal lines whereas genes along the chromosome are represented as rectangle bars. Known MIR169 gene copies are shown as red bars whereas new MIR169 gene copies described in this study are shown as green bars. The bHLH and B-box zinc finger and CCT motif (B-box/CCT) genes are represented as yellow bars. All other protein coding genes in the chromosomal regions under study are represented as black rectangle bars. Orthologous gene pairs are indicated as lines connecting bars, with red color indicating orthology between MIR169 gene pairs and yellow lines indicating orthology between bHLH and B-box/CCT gene pairs respectively. All other orthology between rice and sorghum protein coding genes are indicated as black lines connecting black bars. The physical distance between bHLH and B-box/CCT genes and/or between bHLH or B-Box/CCT genes to the flanking MIR169 copy is indicated. In order to provide a scale of the chromosomal segments highlighted in the figure, the physical distance between the first and the last gene in the segment is indicated and thus serves as a reference to observe expansion and contraction of genomic regions. An inversion event on sorghum chr7 containing the MIR169 cluster occurred relative to the orthologous regions on sorghum chr2 and rice chr8 and chr9 respectively.

FIG. 3. Stem-loop precursor sequences of newly predicted MIR169 copies in rice, sorghum, foxtail millet and maize. The genomic location for each MIR169 stem-loop precursor is given. The predicted mature miR169 sequence is indicated with a red bar. SEQ ID NOs: 1-18 are provided, from top to bottom.

FIG. 4. Sequence alignment of sorghum chr7 segment containing MIR169 gene cluster to homoeologous chromosomal segments from maize. Sorghum sbi-MIR169r/s, sbi-MIR1691 and sbi-MIR169m genes on chr7 are orthologous to maize zma-MIR169e/h; zma-MIR169d and zma-MIR169i respectively on chr4. Notice that the MIR169 cluster on the homoelogous region on maize chr1 was deleted although its flanking genes remained. The orthologous copy of sorghum B-box/CCT gene flanking the MIR169 gene cluster was lost on maize chr4 but retained on the homoelogous segment on chr1. Expansion in the maize genome relative to sorghum is clear when regions on maize chr1 and sorghum chr7 are compared. The region on sorghum chr7 is inverted relative to maize.

FIG. 5. Sequence alignment of sorghum MIR169 cluster on chr1 with orthologous regions from Brachypodium, rice and foxtail millet. The sbi-MIR1690 copy in sorghum allowed the identification of the orthologous osa-MIR169r copy in rice and sit-MIR1690 copy in foxtail millet respectively. For the region containing sbi-MIR169o/t/u on chr1, we could not find sufficient conservation of synteny to identify an orthologous region in sorghum, thus a synteny graph is only shown with sorghum chr1. An inversion event on rice chr3 occurred relative to Brachypodium, foxtail millet and sorghum.

FIG. 6. Sequence alignment of sorghum MIR169 cluster on chr1 with orthologous regions from maize. Sorghum sbi-MIR169u and maize zma-MIR1691 are orthologous copies. There isn't any orthologous MIR169 copy on maize homoeologous chr5. The region on maize chr1 is expanded (comprising a total of 257.6 Kbp) relative to the homoeologous region on chr5 (comprising 18.09 Kbp only). An inversion event occurred on maize homeologous region on chr1.

FIG. 7. Sequence alignment of sorghum MIR169 cluster on chr2 with orthologous regions from maize. Sorghum MIR169 gene cluster on chr2 is collinear with a region on maize chr7 that contains zma-MIR169k, and with the homeologous region on maize chr2 that contains the previously annotated zma-MIR169j and the new copy zma-MIR169s that is described in this study. Although the MIR169 gene cluster on maize chr2 is physically adjacent to the bHLH gene, similarly with the MIR169 gene cluster on sorghum chr2, the homeologous region containing zma-MIR169k lacked the bHLH gene copy. An inversion event on maize chr7 occurred relative to its homeologous region on chr2 and to sorghum chr2.

FIG. 8. Sequence alignment of sorghum MIR169 cluster on chr7 with orthologous regions from Brachypodium, rice and foxtail millet. Rice and sorghum MIR169 gene copies were used to identify and annotate five MIR169 genes in foxtail millet (shown in green). The bHLH and B-box/CCT genes were physically adjacent to MIR169 gene copies in the four species examined. The region examined on sorghum chr7 expanded relative to the orthologous region from the other three grasses and was inverted only in sorghum.

FIG. 9. Sequence alignment of sorghum MIR169 cluster on chr2 with orthologous regions from Brachypodium, rice and foxtail millet. MIR169 gene copies were deleted from Brachypodium chr4 but the flanking genes remained. The MIR169 gene cluster in rice was composed of two copies whereas in sorghum and foxtail millet the cluster comprised three copies. The bHLH gene was present in all four grasses and was physically adjacent to MIR169 gene copies in rice, sorghum and foxtail millet. Sorghum MIR169 gene copies were used to identify and annotate the orthologous copies on foxtail millet scaffold 2 (shown in green).

FIG. 10. Gains and losses of MIR169 gene copies during grass evolution. (A) Phylogenetic distribution of MIR169 gene copies in ancestral and current species with gain and losses of MIR169 copy number during grass evolution. Numbers in squares represent the number of MIR169 gene copies for a given cluster in each species. Numbers along each line represent gains (+) and losses (−) of MIR169 gene copies. The estimated divergence time for each species is given at each node in the tree according to (Bennetzen, et al. 2012; Initiative 2010; Paterson, et al. 2009; Zhang, et al. 2012). The gain in MIR169 copy number of sorghum relative to Brachypodium is depicted. WGD: whole genome duplication; mya: million years ago. Note: WGD in maize is used as a term to represent the allotetraiplody event that took place. (B-D) Neighbor Joining (NJ) phylogenetic trees with boostrap support are shown depicting the relationships of MIR169 stem-loop sequences from the grass species shown in FIG. 6A. (B) NJ phylogenetic tree with Brachypodium (bdi) and rice (osa) MIR169 stem-loop sequences orthologous to sorghum MIR169 copies on chromosome 7. (C) NJ phylogenetic tree with rice (osa) and foxtail millet (sit) MIR169 stem-loop sequences (top) and rice, foxtail millet, sorghum (sbi) and maize (zma) MIR169 stem loop sequences (bottom) orthologous to MIR169 copies on sorghum chromosome 2. (D) NJ phylogenetic tree depicting the relationship of foxtail millet and maize MIR169 copies orthologous to sorghum MIR169 copies on chromosome 1 (top), and Brachypodium, rice, foxtail millet and maize MIR169 copies orthologous to sorghum MIR169 copies on chromosome 1 (bottom).

FIG. 11. Experimental validation of predicted MIR169 stem-loop precursors in sorghum and maize. (A) Sorghum stem-derived small RNAs were mapped to sbi-MIR169t (SEQ ID NO: 19), sbi-MIR169u (SEQ ID NO: 20), and sbi-MIR169v (SEQ ID NO: 21) stem-loop sequences. Only sequences with perfect match to the BTx623 genome are shown. Predicted mature and star miR169 sequence is highlighted in capital letters on the stem-loop sequence. To the left side of each small RNA sequence a label is shown with information about the small RNA library from which it was sequenced (bc01: Mix library; bc02: BTx623 library; bc03: Rio library; bc04: low Brix and early flowering F2 library; bc05: high Brix and late flowering F2 library), together with the abundance of the small RNA read indicated by a number. For sbi-MIR169t, left column, the sequences are positions 2-18; 3-19; 2-21; 3-19; 2-18; 2-18; 2-19; 2-21; 1-20; 3-19; 2-19; 2-20; 1-20; 2-21; 2-22; 1-21; 1-22; 1-23; 1-25; 1-22; 23-41; 27-48; 23-45; 23-39; 23-40; 23-43; 23-45; 23-43; and 23-42 of SEQ ID NO: 19, from top to bottom. For sbi-MIR169t, right column, the sequences are 92-108; 92-109; 92-110; 92-111; 92-110; 92-111; 92-109; 93-110; 92-108; 92-110; 92-111; 90-106; 92-108; 92-109; 94-111; 92-110; and 92-111 of SEQ ID NO: 19, from top to bottom. For sbi-MIR169u, left column, the sequences are positions 16-35; 16-32; 16-33; 16-35; 16-32; 16-33; and 16-35 of SEQ ID NO: 20, from top to bottom. For sbi-MIR169u, right column, the sequences are positions 111-127; 110-126; 110-127; 110-128; 110-128; 110-130; 110-126; 110-126; 111-127; 110-127; 111-128; 110-128; 111-129; 110-129; 112-131; 110-130; 106-127; 111-132; 110-126; 111-127; 110-127; 111-128; 110-128; 111-130; and 111-132 of SEQ ID NO: 20, from top to bottom. For sbi-MIR169v, left column, the sequences are positions 22-38; 22-38; 22-38; 22-42; 22-38; 22-40; 22-42; 5-28; and 12-28 of SEQ ID NO: 21, from top to bottom. For sbi-MIR169v, right column, the sequences are positions 83-100; 84-100; 93-110; and 62-78 of SEQ ID NO: 21, from top to bottom. (B) Maize endosperm-derived small RNAs were mapped to predicted stem-loop precursor zma-MIR169s (SEQ ID NO: 22). For the left column, the sequences are positions 21-44; 22-45; 22-40; 21-44; and 4-28 of SEQ ID NO: 22, from top to bottom. For the right column, the sequences are positions 59-83 and 74-97 of SEQ ID NO: 22, from top to bottom.

FIG. 12. Antisense MIR169r/s gene pair generates small RNAs. Although sequencing of stem-derived small RNAs from grain and sweet sorghum were previously described [10], we mapped small RNAs from our sequenced libraries to the newly annotated sbi-MIR169r and sbi-MIR169s hairpin structures. (A) The most abundant small RNA reads mapped to sbi-MIR169r (SEQ ID NO: 23) corresponded to the miR169r* sequence, whereas the most abundant small RNA reads mapped to sbi-MIR169s (SEQ ID NO: 24) corresponded to miR169s, respectively. For sbi-MIR169r, left column, the sequences are positions 18-37; 19-37; 19-37; 19-37; and 20-37 of SEQ ID NO: 23, from top to bottom. For sbi-MIR169r, right column, the sequences are positions 87-107; 88-106; 88-107; 88-107; 88-105; 89-107; 89-107; 89-107; 89-107; and 90-107 of SEQ ID NO: 23, from top to bottom. For sbi-MIR169s, left column, the sequences are positions 20-40; 21-38; 21-40; 22-39; and 23-40 of SEQ ID NO: 24, from top to bottom. For sbi-MIR169s, right column, the sequences are positions 90-107; 90-107; and 90-109 of SEQ ID NO: 24, from top to bottom. (B) Nucleotide polymorphism between miR169r* (SEQ ID NO: 25) and miR169s (SEQ ID NO: 26).

FIG. 13. List of predicted targets of sbi-miR169r*. The psRNATarget program was used to predict mRNAs targeted by sbi-miR169r*. The miR169r*-target alignment is shown together with the expectation level of the prediction with 1 as high confident and 3.5 less confident. The annotation for each predicted gene is shown in conjunction with the region where the miR169r* recognition sequence is located (exon or 3′UTR). Sequences in FIG. 13A are, from top to bottom: SEQ ID NO: 27; SEQ ID NO: 31; SEQ ID NO: 27; SEQ ID NO: 32; SEQ ID NO: 27; SEQ ID NO: 33; SEQ ID NO: 28; SEQ ID NO: 34; SEQ ID NO: 29; SEQ ID NO: 35; SEQ ID NO: 28; SEQ ID NO: 36; SEQ ID NO: 27; SEQ ID NO: 37; SEQ ID NO: 30; SEQ ID NO: 38; SEQ ID NO: 30; and SEQ ID NO: 39. Sequences in FIG. 13B are, from top to bottom: SEQ ID NO: 30; SEQ ID NO: 40; SEQ ID NO: 30; SEQ ID NO: 41; SEQ ID NO: 30; SEQ ID NO: 42; SEQ ID NO: 29; SEQ ID NO: 43; SEQ ID NO: 29; SEQ ID NO: 44; SEQ ID NO: 29; SEQ ID NO: 45; SEQ ID NO: 29; SEQ ID NO: 46; SEQ ID NO: 30; SEQ ID NO: 47; SEQ ID NO: 30; and SEQ ID NO: 48.

FIG. 14. List of predicted targets of sbi-miR169s. The psRNATarget program was used to predict mRNAs targeted by sbi-miR169s. The miR169s-target alignment is shown together with the expectation level of the prediction with 1 as high confident and 3.5 less confident. The annotation for each predicted gene is shown in conjunction with the region where the miR169s recognition sequence is located (exon or 3′UTR). Sequences are, from top to bottom: SEQ ID NO: 49; SEQ ID NO: 49; SEQ ID NO: 51; SEQ ID NO: 49; SEQ ID NO: 52; SEQ ID NO: 50; SEQ ID NO: 53; SEQ ID NO: 50; SEQ ID NO: 54; SEQ ID NO: 49; and SEQ ID NO: 55.

FIG. 15. Sequence alignment of sorghum MIR169 cluster on chr7 with orthologous regions from Brachypodium, soybean and cassava. There is conservation of synteny between monocot species Brachypodium and sorghum and dicot species soybean and cassava when chromosomal segments containing MIR169 gene copies and their flanking genes are aligned. Conservation of synteny allowed the identification of new MIR169 gene copies on soybean chromosome 6 (gma-MIR169w) and cassava scaffold 01701 (mes-MIR169w), respectively. Physical association on the chromosome between MIR169 and the flanking bHLH gene was retained in soybean and cassava as well. Notice the inversion on soybean chr6.

FIG. 16. Sequence alignment of sorghum MIR169 cluster on chr2 with orthologous regions from Brachypodium, soybean and cassava. The alignment of sorghum MIR169 cluster on chr2 with soybean chr8 and cassava scaffold 09876 allowed the identification of two new MIR169 gene copies in soybean (gma-MIR169x and gma-MIR169y) and one new copy in cassava (mes-MIR169y), respectively. The physical association of MIR169 gene copies with the bHLH was retained in soybean and cassava. An inversion occurred on soybean chr8.

FIG. 17. Conservation of synteny between sorghum and grapevine chromosomal segments containing MIR169 gene copies. Sorghum segments containing MIR169 gene clusters from chr2 and chr7 were aligned to the grapevine genome based on orthologous gene pairs. Because grapevine is a hexopaleo-polyploid, we found a 2:3 chromosomal relationship between sorghum and grapevine. Colinearity allowed the identification of a new MIR169 copy (vvi-MIR169z) in grapevine chr14. Different grapevine chromosomes are represented in colors whereas sorghum chromosomes are in black. Relative to sorghum chr2, grapevine had in inversion event on chr14 and chr17. The association of MIR169 with its flanking COL gene was maintained on grapevine chr14 and chr1 whereas the association of MIR169 with the bHLH gene was maintained on chr1.

FIG. 18. Sub-functionalization of Brachypodium bHLH gene copy. (A) Left: Neighbor Joining (NJ) phylogenetic tree of orthologous bHLH proteins with the Arabidopsis bHLH137 protein as reference. Middle: a representation of the gene structure in exons (boxes) and introns (lines) (5′ and 3′ UTRs not included). Right: graphic representation of the linear protein with the bHLH domain represented as an orange box and the HLH domain as a yellow box with orange border. (B) Protein alignment highlighting the bHLH motif with AtbHLH137 protein as reference. The Brachypodium protein encoded by the gene Bradi4g34870 lost most of the basic domain, becoming a HLH protein instead. Sequences are, from top to bottom, SEQ ID NOs: 56-72. (C) Graph depicting the average synonymous and non-synonymous substitution rate of the bHLH Bradi3g41510 orthologous gene pairs compared to HLH Bradi4g34870 orthologous gene pairs.

FIG. 19. Evolution of the Zinc finger, B-box and CCT domain protein. (A) Left: Neighbor Joining (NJ) phylogenetic tree of B-box and CCT motif orthologous proteins with Arabidopsis COL14 protein as reference. Center: graphic representation of the B-box and CCT motif gene structure for each species with exons as boxes and introns as lines (5′ and 3′ UTRs not shown). Right: linear representation of the B-box and CCT motif protein for each species with the Zinc finger, B-box domain shown as a blue box where the CCT domain is shown as a red box. (B) Protein alignment highlighting the Zinc finger, B-box domain in blue boxes (Arabidopsis COL14 has two) and the CCT domain in a red box. Sequences are, from top to bottom, SEQ ID NOs: 73-80.

FIG. 20. The “Drought and Flowering Genetic Module Hypothesis”. Here we suggest that trade-offs between drought stress and flowering time could be explained in part by genetic linkage of MIR169 and COL genes. In this model, a given COL gene genetically linked to a MIR169 gene will be positively selected over any other COL gene located somewhere else in the genome. This is so because COL proteins can replace the NF-YA (HAP2) subunit from the NF-YA, NF-YB (HAP3) and NF-YC (HAPS) hetero-trimeric transcription factor complex [26], with NF-YA mRNA targeted by miR169 [38]. Thus, depending on water availability, plants can adjust their flowering time according to the severity of drought during the growing season by modulating the expression of miR169 and COL genes. Under this scenario, high miR169 expression lower NF-YA mRNA levels, consequently decreasing NF-YA protein levels, which may increase the frequency of COL protein to interact with NF-YB and NF-YC subunits and thus guide the transcription complex toward the expression of CCAAT box genes involved in flowering. The current model establishes a genetic framework to explain the observation that plants flower early under drought compared to well watered environments [39].

### DETAILED DESCRIPTION OF THE INVENTION

Expansion and contraction of microRNA families can be studied in sequenced plant genomes through sequence alignments. Here we focused on miR169 in sorghum because of its implications in drought tolerance and stem sugar content. We were able to discover many miR169 copies that have escaped standard genome annotation methods. A new miR169 cluster was found on sorghum chromosome 1 (chr1). This cluster is composed of the previously annotated sbi-MIR1690 together with two newly found MIR169 copies, named sbi-MIR169t and sbi-MIR169u. We also found that a miR169 cluster on sorghum chr7 consisting of sbi-MIR1691, sbi-MIR169m, and sbi-MIR169n is contained within a chromosomal inversion of at least 500 Kbp that occurred in sorghum relative to Brachypodium, rice, Foxtail millet and maize. Surprisingly, synteny of chromosomal segments containing MIR169 copies with linked bHLH and CONSTANS-LIKE genes extended from Brachypodium to dictotyledonous species such as grapevine, soybean, and cassava, indicating a strong conservation of linkages of certain flowering and/or plant height genes and microRNAs, which may explain linkage drag of drought and flowering traits and would have consequences for breeding new varieties. Furthermore, alignment of rice and sorghum orthologous regions revealed the presence of two additional miR169 gene copies (miR169r and miR169s) on sorghum chr7 that formed an antisense miRNA gene pair. Both copies are expressed and target different set of genes. Synteny-based analysis of microRNAs among different plant species should lead to the discovery of new microRNAs in general and contribute to our understanding of their evolution.

### I. DEFINITIONS

The following definitions are provided to facilitate an understanding of the present invention. Unless defined otherwise, all technical and scientific terms used herein have the same meaning as commonly understood by one of ordinary skill in the art to which this invention belongs. Generally, conventional methods of molecular biology, microbiology, recombinant DNA techniques, cell biology, and virology within the skill of the art are employed in the present invention. Such techniques are explained fully in the literature, see, e.g., Maniatis, Fritsch & Sambrook, Molecular Cloning: A Laboratory Manual (1982); DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. (1984)); Animal Cell Culture (R. I. Freshney, ed. 1986); and RNA Viruses: A Practical Approach, (Alan, J. Cann, Ed., Oxford University Press, 2000).

For purposes of the invention, “Nucleic acid”, “nucleotide sequence” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

According to the present invention, an isolated or biologically pure molecule or cell is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route. The term “promoter” or “promoter region” generally refers to the transcriptional regulatory regions of a gene. The “promoter region” may be found at the 5′ or 3′ side of the coding region, or within the coding region, or within introns. Typically, the “promoter region” is a nucleic acid sequence which is usually found upstream (5′) to a coding sequence and which directs transcription of the nucleic acid sequence into mRNA. The “promoter region” typically provides a recognition site for RNA polymerase and the other factors necessary for proper initiation of transcription.

Promoters useful in some embodiments of the present invention may be tissue-specific or cell-specific. The term “tissue-specific” as it applies to a promoter refers to a promoter that is capable of directing selective expression of a nucleotide sequence of interest to a specific type of tissue in the relative absence of expression of the same nucleotide sequence of interest in a different type of tissue (e.g., flower vs. root). The term “cell-specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell-specific” when applied to a promoter also means a promoter capable of promoting selective expression of a nucleotide sequence of interest in a region within a single tissue. Alternatively, promoters may be constitutive or regulatable. Additionally, promoters may be modified so as to possess different specificities.

The term “vector” relates to a single or double stranded circular nucleic acid molecule that can be infected, transfected or transformed into cells and replicate independently or within the host cell genome. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. An “expression vector” is a specialized vector that contains a gene or nucleic acid sequence with the necessary regulatory regions needed for expression in a host cell.

DNA constructs or vectors of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al., Embo J. 3:2717-2722 (1984). Electroporation techniques are described in Fromm et al., Proc. Natl. Acad. Sci. USA 82:5824 (1985). Ballistic transformation techniques are described in Klein et al., Nature 327:70-73 (1987).

Agrobacterium tumefaciens-mediated transformation techniques, including disarming and use of binary vectors, are well described in the scientific literature. See, for example, Horsch et al., Science 233:496-498 (1984), and Fraley et al., Proc. Natl. Acad. Sci. USA 80:4803 (1983).

Transformed plant cells that are derived by any of the above transformation techniques can be cultured to regenerate a whole plant that possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al., Ann. Rev. of Plant Phys. 38:467-486 (1987).

One of skill will recognize that after the expression cassette or vector is stably incorporated in transgenic plants and confirmed to be operable, it can be introduced into other plants by sexual crossing. Any of a number of standard breeding techniques can be used, depending upon the species to be crossed.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The terms “miRNA” and “microRNA” refer to about 10-35 nt, preferably about 15-30 nt, and more preferably about 19-26 nt, non-coding RNAs derived from endogenous genes encoded in the genomes of plants and animals. They are processed from longer hairpin-like precursors termed pre-miRNAs that are often hundreds of nucleotides in length. MicroRNAs assemble in complexes termed miRNPs and recognize their targets by antisense complementarity. These highly conserved, endogenously expressed RNAs are believed to regulate the expression of genes by binding to the 3′-untranslated regions (3′-UTR) of specific mRNAs as well as other regions on targeted mRNAs. Without being bound by theory, a possible mechanism of action assumes that if the microRNAs match 100% their target, i.e. the complementarity is complete, the target mRNA is cleaved, and the miRNA acts like a siRNA. However, if the match is incomplete, i.e. the complementarity is partial, then the translation of the target mRNA is blocked. The manner by which a miRNA base-pairs with its mRNA target correlates with its function: if the complementarity between a mRNA and its target is extensive, the RNA target is cleaved; if the complementarity is partial, the stability of the target mRNA in not affected but its translation is repressed.

The term “RNA interference” or “RNAi” refers generally to a process or system in which a RNA molecule changes the expression of a nucleic acid sequence with which RNA molecule shares substantial or total homology. The term “RNAi agent” refers to an RNA sequence that elicits RNAi.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting different target miRNAs may be between 15-35 nucleotides in length.

“Pri-miRNAs” are several hundred to thousands of base pairs in size. Pri-miRNA contains at least 1, and up to 6, nucleotide hairpin loop structures when transcribed from polycistronic units. They can be composed of multiple miRNAs, and in a particular arrangement of the invention five miRNAs are processed from one nucleic acid sequence. These sequences can also contain siRNA nucleic acids that repress gene transcription once processed in the RNAi system.

As used herein, “agricultural formulations” include formulations for use in the field. The phrase “agriculturally acceptable formulation” as used herein refers to a composition or formulation that allows for the effective distribution of the nucleic acid molecules of the instant invention in the physical location most suitable for their desired activity.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxiliary agent or vehicle with which an active agent of the present invention is administered. Agriculturally acceptable carriers can be sterile liquids, such as water and oils, including those of petroleum, animal, vegetable or synthetic origin. Water or aqueous saline solutions and aqueous dextrose and glycerol solutions are preferably employed as carriers.

With respect to single-stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (see Sambrook et al. (2001) Molecular Cloning. A Laboratory Manual, Cold Spring Harbor, N.Y.: Cold Spring Harbor Laboratory Press):

Tₘ=81.5° C.+16.6 Log [Na+]+0.41 (% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. Depending upon the specific sequence involved, the Tₘ of a DNA duplex decreases by 0.5-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high-stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

“Corresponding” means identical to or complementary to the designated sequence. The sequence may be generated in any manner, including chemical synthesis, DNA replication, reverse transcription or a combination thereof. Being “Complementary” means that a nucleic acid, such as DNA and RNA, encodes the only corresponding base pair that non-covalently connects sequences by two or three hydrogen bonds. There is only one complementary base for any of the bases found in DNA and in RNA, and skilled artisans can reconstruct a complementary strand for any single stranded nucleic acid.

The present invention also includes active portions, fragments, derivatives and functional or non-functional mimetics of the miRNAs of the invention. A “fragment” or “portion” of a sequence means a stretch of residues of at least about five to seven contiguous residues, often at least about seven to nine contiguous residues, typically at least about nine to fifteen contiguous residues and, most preferably, at least about fourteen or more contiguous residues.

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

A “derivative” of a polypeptide, polynucleotide or fragments thereof means a sequence modified by varying the sequence of the construct, e.g. by manipulation of the nucleic acid encoding the protein or by altering the protein itself. “Derivatives” of a gene or nucleotide sequence refers to any isolated nucleic acid molecule that contains significant sequence similarity to the gene or nucleotide sequence or a part thereof. In addition, “derivatives” include such isolated nucleic acids containing modified nucleotides or mimetics of naturally-occurring nucleotides.

The term “functional” as used herein implies that the nucleic or amino acid sequence is functional for the recited assay or purpose.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide can depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein. The term “gene” refers to a nucleic acid comprising an open reading frame encoding a polypeptide, including both exon and (optionally) intron sequences. The nucleic acid may also optionally include non coding sequences such as promoter or enhancer sequences. The term “intron” refers to a DNA sequence present in a given gene that is not translated into protein and is generally found between exons.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, depending on the complexity of the target sequence, the oligonucleotide probe typically contains about 10-50 or more nucleotides, more preferably, about 15-25 nucleotides.

The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The terms “percent similarity”, “percent identity” and “percent homology” when referring to a particular sequence are used as set forth in the University of Wisconsin GCG software program.

The term “delivery” as used herein refers to the introduction of foreign molecule (i.e., miRNA containing nanoparticle) into cells. The term “administration” as used herein means the introduction of a foreign molecule into a cell. The term is intended to be synonymous with the term “delivery”.

The term “kit” refers to a combination of reagents and other materials.

### II. USES OF MIRNA CONSTRUCTS

The present invention is based, at least in part, on the identification of new miRNAs in sorghum. The nucleic acids of the invention can be used to control gene expression in plants. In some embodiments, the expression cassettes encoding the miRNAs of the invention are prepared and introduced into plants. The encoded miRNAs then control expression of the endogenous target genes. Alternatively, one can modify the target gene so as to render it miRNA-resistant by modifying the sequence to decrease or inhibit pairing with the miRNA. The modifications will typically be selected such that the sequence of the encoded protein is not altered. The modified target gene can be incorporated into an expression cassette and introduced into a plant. Alternatively, an endogenous target gene can be modified using known techniques (e.g., homologous recombination).

Nucleic acid molecules encoding the miRNAs of the invention may be prepared by using recombinant DNA technology methods. The availability of nucleotide sequence information enables preparation of nucleic acid-based molecules of the invention by a variety of means. The RNAs may be used for a variety of purposes in accordance with the present invention. In a preferred embodiment of the invention, a nucleic acid delivery vehicle (i.e., an expression vector) for modulating target gene expression is provided wherein the expression vector comprises a nucleic acid sequence coding at least one miRNA, or a functional fragments thereof as described herein. Administration of miRNA or derivatives thereof encoding expression vectors to a plant results in the modulation of target gene expression, particularly genes involved in sugar metabolism and flowering.

For some applications, an expression construct may further comprise regulatory elements which serve to drive expression in a particular cell or tissue type. Such regulatory elements are known to those of skill in the art and discussed in depth in Sambrook et al. (1989) and Ausubel et al. (1992). The incorporation of tissue specific regulatory elements in the expression constructs of the present invention provides for at least partial tissue tropism for the expression of miRNA(s). For example, the miRNA constructs can be subcloned into a vector downstream of a tissue specific promoter/enhancer to target gene expression in a particular region of the plant (e.g., root, vs. leaves).

### III. AGRICULTURAL COMPOSITIONS

The expression vectors of the present invention may be incorporated into agricultural compositions that may be delivered to a plant. In a particular embodiment of the present invention, compositions comprising isolated nucleic acids which enable the recipient to produce biologically effective miRNAs that modulate target gene expression in the recipient plant are provided. Herein we describe a broad spectrum of the small RNA component of the sorghum transcriptome and provide new insights into how complex processes like carbohydrate metabolism and flowering time are regulated at the post-transcriptional level. Elucidation of this regulatory process provides an opportunity to improve biofuel production, for example, by increasing stem sugar rather than cellulose and increasing biomass because of delayed flowering (38). The compositions may be administered alone or in combination with at least one other agent, such as a stabilizing compound, which may be administered in any sterile, biocompatible carrier, including, but not limited to, saline, buffered saline, dextrose, and water. In preferred embodiments, the pharmaceutical compositions also contain a agriculturally acceptable excipient. Acceptable excipients include, but are not limited to, liquids such as water, saline, glycerol, sugars and ethanol.

After agricultural compositions have been prepared, they may be placed in an appropriate container or kit and labeled for use. For administration of miRNA-containing vectors, such labeling would include amount, frequency, and method of delivery.

### IV. KITS AND ARTICLES OF MANUFACTURE

Any of the aforementioned compositions or methods can be incorporated into a kit which may contain at least one miRNA sequence or a polycistronic transcript of multiple miRNAs. If the agricultural composition in liquid form is under risk of being subjected to conditions which will compromise the stability of the miRNAs or vectors encoding the same, it may be preferred to produce the finished product containing the miRNAs in a solid form, e.g. as a freeze dried material, and store the product is such solid form. The product may then be reconstituted (e.g. dissolved or suspended) in a saline or in a buffered saline ready for use prior to administration.

Hence, the present invention provides a kit comprising (a) a first component containing miRNAs as defined hereinabove, optionally in solid form, and (b) a second component containing saline or a buffer solution (e.g. buffered saline) adapted for reconstitution (e.g. dissolution or suspension) or delivery of said miRNAs or a vector encoding the same. Preferably said saline or buffered saline has a pH in the range of 4.0-8.5, and a molarity of 20-2000 mM. In a preferred embodiment the saline or buffered saline has a pH of 6.0-8.0 and a molarity of 100-500 mM. In a most preferred embodiment the saline or buffered saline has a pH of 7.0-8.0 and a molarity of 120-250 mM.

### VI. AGRICULTURAL APPLICATIONS

As mentioned previously, a preferred embodiment of the invention comprises delivery of at least one vector encoding an miRNA or a polycistronic miRNA transcript to a plant to control flowering and/or sugar metabolism. Alternatively, inhibitors of the miRNAs which interfere with the functions of the miRNAs disclosed herein may be delivered to target plants of interest. Field trials can be designed to assess the safety, tolerability, pharmacokinetics, and pharmacodynamics of the miRNA constructs of the invention.

The following materials and methods are provided to facilitate practice of the present invention.

### DNA Sequences

Rice sequences were downloaded from the Rice Annotation Project Database (RAP-DB) website (http://rapdb.dna.affrc.gojp/), whereas Brachypodium, foxtail millet, sorghum, maize, grapevine, soybean and cassava sequences were downloaded from the Join Genome Institute (JGI) website (www.phytozome.net). MicroRNA sequences were downloaded from the miRBase database (http://www.mirbase.org/).

### MIR169 Gene Prediction and Annotation

Stem-loop precursors/hairpin structures from previously annotated MIR169 genes were used in reciprocal Blastn analysis during the process of creating synteny graphs. Previously known MIR169 stem-loop precursors were used as query sequences with Blastn. When the corresponding target sequences identified matched a genomic region where there was no any previous annotation of a MIR169 gene copy, we took a 100-300 bp segment and fed it into an RNA folding program (RNAfold web server: http://rna.tbi.univie.ac.at/cgi-bin/RNAfold.cgi) to look for signatures of hairpin-like structures typical of microRNAs. Guidelines in microRNA gene prediction were followed as suggested by Meyers et al. 2008 (Meyers, et al. 2008).

### Experimental Validation of Predicted MIR169 Genes

We took advantage of our previously sequenced small RNA libraries from sorghum stems (Calvino, et al. 2011) and mapped small RNAs to the newly predicted MIR169r/s/t/u/v hairpin sequences. To validate the newly predicted MIR169s in maize, we used the SOLiD platform to sequence small RNAs derived from endosperm tissue from B73 and Mo17 inbred lines as well as endosperm tissue derived from their reciprocal crosses. Small RNA reads were then mapped to zma-MIR169s stem loop precursor.

Prediction of miR169 Targets

Target prediction was conducted in sorghum for the newly discovered miR169r* and miR169s microRNAs using the Small RNA Target Analysis Server psRNATarget (Dai and Zhao 2011) at http://plantgrn.noble.org/psRNATarget/. In addition to the sorghum genome sequence incorporated into psRNATarget (Sorghum DCFI Gene Index SBGI Release 9) as preloaded transcripts, we also uploaded a FASTA file from phytozome on the world wide web at phytozome.net/dataUsagePolicy.php?org=Org_Sbicolor, with all sorghum genes coding sequences and used this data set for target prediction as well. Target prediction was conducted for the annotated 21 nt miR169 as well as for the most abundant small RNA reads different from 21 nt in size that matched the predicted miR169 sequence (miR169 variants).

### Estimation of MIR169 Gene Number in Ancestral Species

In order to estimate the numbers of MIR169 genes in ancestral species of the grass family together with gains and losses of MIR169 copies during grass evolution, we took the parsimony approach as described previously by Nozawa and colleagues (Nozawa, et al. 2012).

### Estimation of Substitution Rates in MIR169 Genes and Ancient Duplication Time

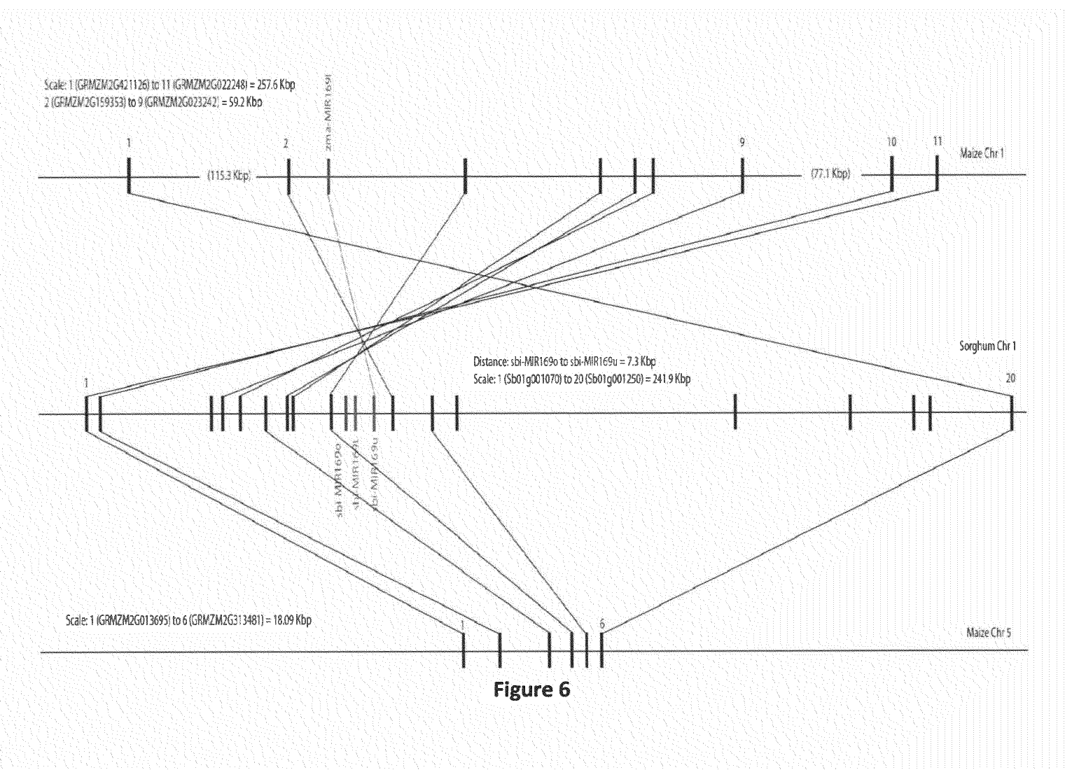

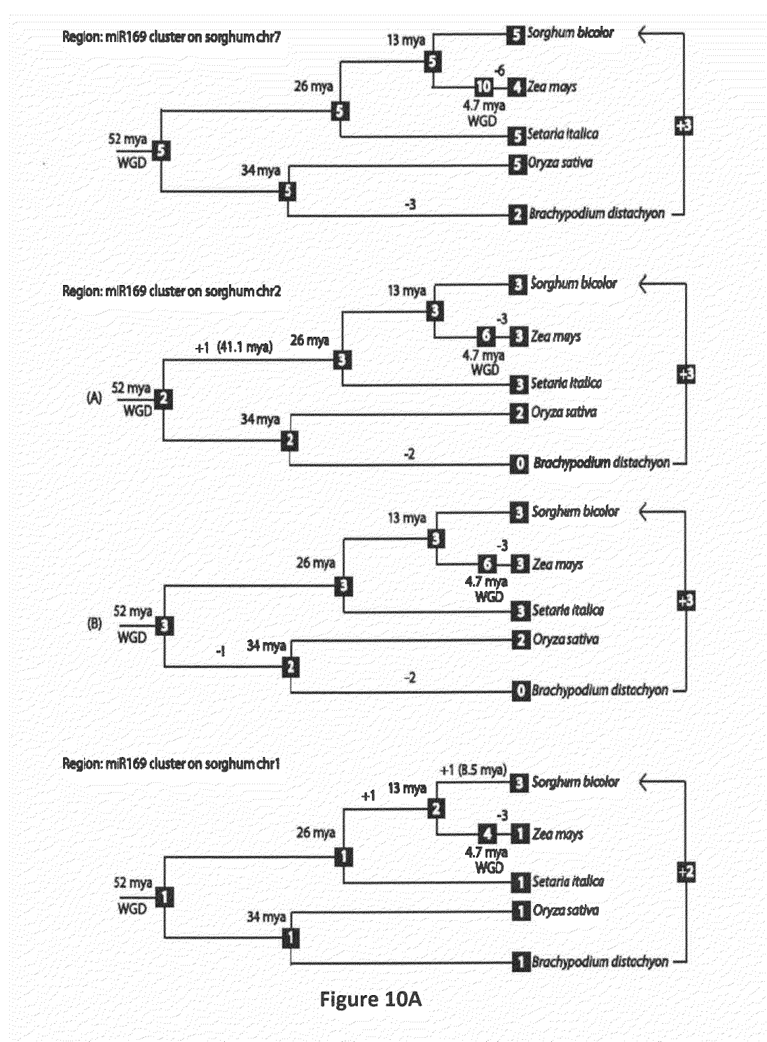

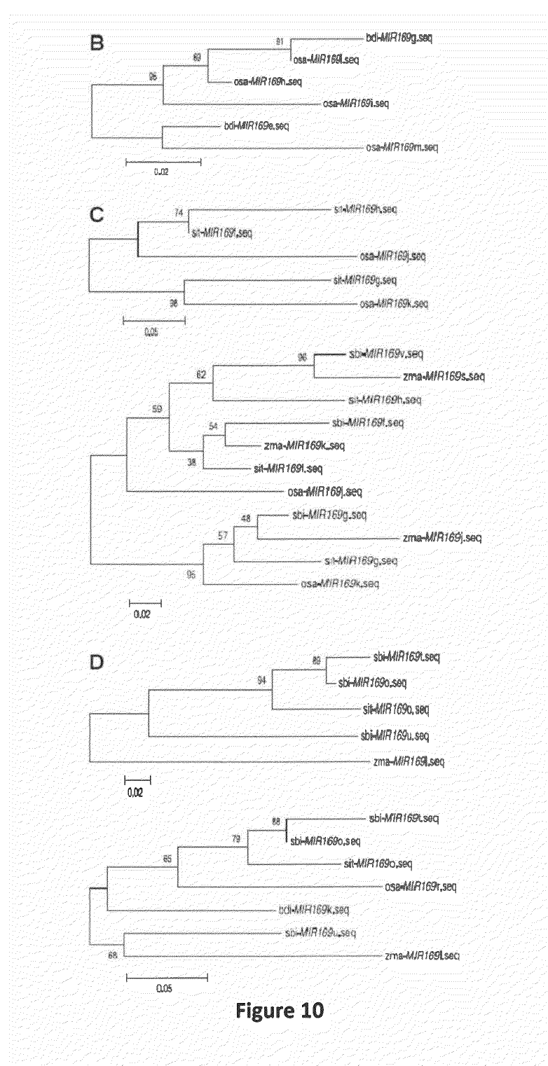

To study the rate of nucleotide substitution in MIR169 genes, we aligned MIR169 stemloop sequences using MUSCLE, available with the MEGA5 software package (Tamura, et al. 2011). When we analyzed the gained MIR169 gene copy that gave rise to sit-MIR169h, sbi-MIR169v and zma-MIR169s copies (FIG. 6A: region miR169 cluster on sorghum chr2), we first computed the average (Jukes and Cantor) distance (Da) between zma-MIR169s/sbi-MIR169v and zma-MIR169s/sit-MIR169h gene pairs. The substitution rate (R) was subsequently calculated with the formula R=Da/2T where T is the divergence time (in this case 26 mya), when the ancestor of maize and sorghum diverged from foxtail millet. We then calculated the ancient duplication time at which sit-MIR169h arose by using the formula t=da/2R, where t is the divergence time of two sequences and da is the average distance between sequences in the miR169 cluster (the average of pairwise distances between sit-MIR169h/sit-MIR169g and sit-MIR169h/sit-MIR169f, respectively). A similar rationale was applied for the calculation of the ancient duplication time of sbi-MIR169t in the sorghum miR169 cluster 1 (FIG. 10A).

Rate of Synonymous and Non-Synonymous Substitutions of the bHLH Orthologous Gene Pairs

We used gene exon sequences to estimate synonymous and non-synonymous substitutions using the MEGA5 program (Tamura, et al. 2011). The synonymous and non-synonymous substitution rate was calculated for a given bHLH orthologous gene pair (Brachypodium-rice; Brachypodium-foxtail millet; Brachypodium-sorghum and Brachypodium-maize), where Brachypodium bHLH gene Bradi3g41510 was compared to the HLH gene Bradi4g34870.

### Phylogenetic Analysis

Phylogenetic analysis were performed by creating multiple alignments of nucleotide or amino acid sequences using MUSCLE and Clustal W, respectively, and phylograms were drawn with the MEGA5 program using the NJ (Neighbor Joining) method (Tamura, et al. 2011). Multiple alignments of microRNA 169 stem-loop sequences were improved by removing the unreliable regions from the alignment using the web-based program GUIDANCE (http://guidance.tau.ac.il), and NJ phylogenetic tress were created with 2000 Boostrap replications and the model/method used was the Maximum Composite Likelihhood.

### Example I

### New MIR169 Gene Copies in the Rice, Sorghum and Maize Genomes

Here, we analyzed the process of tandem duplication that gave rise to MIR169 gene clusters in sorghum (Sorghum bicolor (L.) Moench) and traced its evolutionary path by aligning contiguous chromosomal segments of diploid Brachypodium, rice, foxtail millet, and the two homoeologous regions of allotetraploid maize. We have chosen miR169 as an example because of its possible role in stem-sugar accumulation in sorghum besides its previously described role in drought stress response in several plant species. We discovered allelic variation in miR169 expression between grain and sweet sorghum, suggesting that miR169 could also play a role in the sugar content of sorghum stems (Calvino, et al. 2011). Although high sugar content in stems is a trait shared by sorghum and sugarcane (Calvino, et al. 2008; Calvino, et al. 2009), this trait seems to be silent in other grasses (Calvino and Messing 2011). This prompted us to investigate the evolution and dynamic amplification of miR169 gene copies in grass genomes. We found that synteny of chromosomal segments containing MIR169 gene copies was conserved between monocotyledoneous species such as Brachypodium and sorghum but surprisingly also across the monocot barrier in dicotyledoneous species such as grapevine, soybean, and cassava. Furthermore, linkage of MIR169 copies with a bHLH gene similar to Arabidopsis bHLH137 and with a CONSTANS-LIKE gene similar to Arabidopsis COL14 was conserved in all the grasses examined as well as in soybean and cassava (linkage between MIR169 and bHLH genes) and grapevine (linkage between MIR169 and COL14 genes). We discuss the importance of this finding for breeding crops with enhanced bioenergy traits.

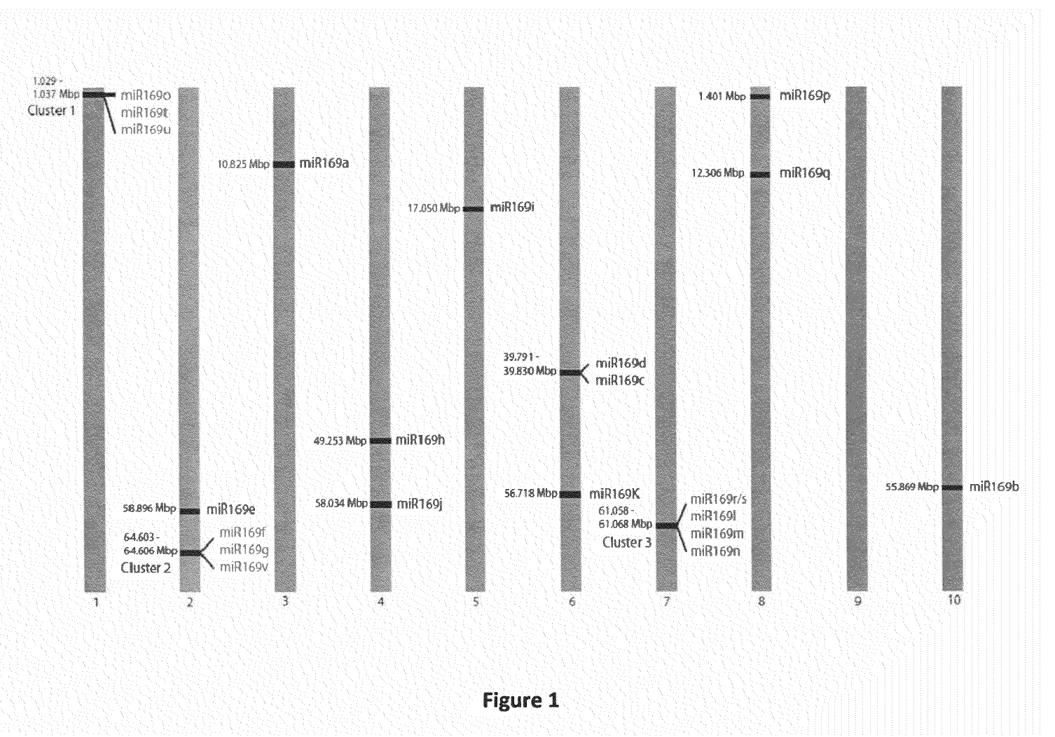

A miRNA cluster as defined in the miRBase database (release 19, August 2012) is composed of two or more miRNA gene copies that are located on the same chromosome and separated from each other by a distance of 10 Kbp or less. The distance set to define a miRNA cluster is arbitrary though, as evidenced by a cluster composed of sixteen copies of MIR2118 distributed over a 18 Kbp segment on rice chr4 (Sun, et al. 2012). The sequencing of the sorghum genome allowed the identification of seventeen MIR169 gene copies, from which five were arranged in two clusters, one located on chr2 (sbi-MIR169f and sbi-MIR169g) and the other located on chr7 (sbi-MIR1691, sbi-MIR169m and sbi-MIR169n, respectively (Paterson, et al. 2009) (FIG. 1; Table 1).

TABLE 1
Summary of MIR169 gene copies described in this study

| Chromosome | Gene ID¹ | Coordinates² | Strand | Distance between genes flanking the cluster³ |
|---|---|---|---|---|
| Brachypodium | | | | |
| distachyon | | | | |
| chr1 | bdi-MIR169k | 1,175,425 . . . 1,175,598 | + | |
| chr3 | bdi-MIR169e | 43,441,526 . . . 43,441,689 | + | Cluster 1: bdi-MIR169e to bdi-MIR169g = 2,960 bp |
| | bdi-MIR169g | 43,444,486 . . . 43,444,666 | + | |
| Oryza sativa | | | | |
| chr3 | osa-MIR169r | 35,782,397 . . . 35,782,553 | + | |
| chr8 | osa-MIR169i | 26,891,154 . . . 26,891,261 | + | Cluster 1: osa-MIR169i to osa-MIR169q = 14,446 bp |
| | osa-MIR169h | 26,895,354 . . . 26,895,475 | + | |
| | osa-MIR169m | 26,901,902 . . . 26,902,039 | + | |
| | osa-M1R169l | 26,905,493 . . . 26,905,600 | + | |
| | osa-MIR169q | 26,905,600 . . . 26,905,493 | − | |
| chr9 | osa-MIR169j | 19,788,861 . . . 19,788,985 | + | Cluster 2: osa-MIR169j to osa-MIR169k = 3,272 bp |
| | osa-MIR169k | 19,792,133 . . . 19,792,288 | + | |
| Setaria italica | | | | |
| chr9 | sit-MIR169o | 526,081 . . . 525,981 | − | |
| chr2 | sit-MIR169f | 36,921,078 . . . 36,921,205 | + | Cluster 1: sit-MIR169f to sit-MIR169h = 3,137 bp |
| | sit-MIR169g | 36,923,991 . . . 36,924,143 | + | |
| | sit-MIR169h | 36,924,215 . . . 36,924,361 | + | |
| chr6 | sit-MIR169i | 33,994,480 . . . 33,994,680 | + | Cluster 2: sit-MIR169i to sit-MIR169s = 8,922 bp |
| | sit-MIR169j | 33,997,832 . . . 33,997,997 | + | |
| | sit-MIR169k | 34,001,008 . . . 34,001,109 | + | |
| | sit-MIR169r | 34,003,536 . . . 34,003,402 | − | |
| | sit-MIR169s | 34,003,402 . . . 34,003,536 | + | |
| Sorghum bicolor | | | | |
| chr1 | sbi-MIR169o | 1,029,916 . . . 1,029,814 | − | Cluster 1: sbi-MIR169o to sbi-MIR169u = 7,321 bp |
| | sbi-MIR169t | 1,030,265 . . . 1,030,155 | − | |
| | sbi-MIR169u | 1,037,237 . . . 1,037,096 | − | |
| chr2 | sbi-MIR169f | 64,603,670 . . . 64,603,817 | + | Cluster 2: sbi-MIR169f to sbi-MIR169v = 3,049 bp |
| | sbi-MIR169g | 64,606,503 . . . 64,606,654 | + | |
| | sbi-MIR169v | 64,606,719 . . . 64,606,868 | + | |
| chr7 | sbi-MIR169r | 61,058,625 . . . 61,058,750 | + | Cluster 3: sbi-MIR169r to sbi-MIR169n = 12,648 bp |
| | sbi-MIR169s | 61,058,750 . . . 61,058,625 | − | |
| | sbi-M1R169l | 61,062,736 . . . 61,062,640 | − | |
| | sbi-MIR169m | 61,068,118 . . . 61,068,027 | − | |
| | sbi-MIR169n | 61,071,181 . . . 61,071,273 | + | |
| Zea mays | | | | |
| chr1 | zma-MIR169l | 298,277,019 . . . 298,277,107 | + | |
| chr2 | zma-MIR169j | 192,700,339 . . . 192,700,489 | + | Cluster 1: zma-MIR169j to zma-MIR169s = 277 bp |
| | zma-MIR169s | 192,700,616 . . . 192,700,748 | + | |
| chr4 | zma-MIR169i | 47,241,963 . . . 47,242,153 | + | Cluster 2: zma-MIR169i to zma-MIR169e = 271,605 bp |
| | zma-MIR169d | 47,454,177 . . . 47,454,304 | − | |
| | zma-MIR169h | 47,513,567 . . . 47,513,694 | + | |
| | zma-MIR169e | 47,513,695 . . . 47,513,568 | − | |
| chr7 | zma-MIR169k | 135,706,179 . . . 135,706,311 | − | |
| Vitis vinifera | | | | |
| chr1 | vvi-MIR169y | 22,233,573 . . . 22,233,820 | + | |
| chr14 | vvi-MIR169z | 25,082,612 . . . 25,082,498 | − | Cluster 1: vvi-MIR169z to vvi-MIR169e = 367 bp |
| | vvi-MIR169e | 25,082,865 . . . 25,082,717 | − | |
| chr17 | vvi-MIR169x | 355,713 . . . 355,837 | − | |
| Glycine max | | | | |
| chr6 | gma-MIR169w | 13,783,352 . . . 13,783,225 | − | |
| chr8 | gma-MIR169x | 717,092 . . . 717226 | + | Cluster 1: gma-MIR169o to gma-MIR169p = 7,248 bp |
| | gma-MIR169y | 724,205 . . . 724,340 | + | |
| Manihot esculenta | | | | |
| scaffold01701 | mes-MIR169w | 436,633 . . . 436,794 | + | |
| scaffold09876 | mes-MIR169y | 536,510 . . . 536,709 | − | |

¹In green color are microRNA genes identified in this study
²Chromosomal positions are based on Phytozome annotation for all the species except rice that is based on RAPDB annotation
³Distance within the cluster is calculated from the beginning of the first miRNA gene to the beginning of the last miRNA gene in the cluster

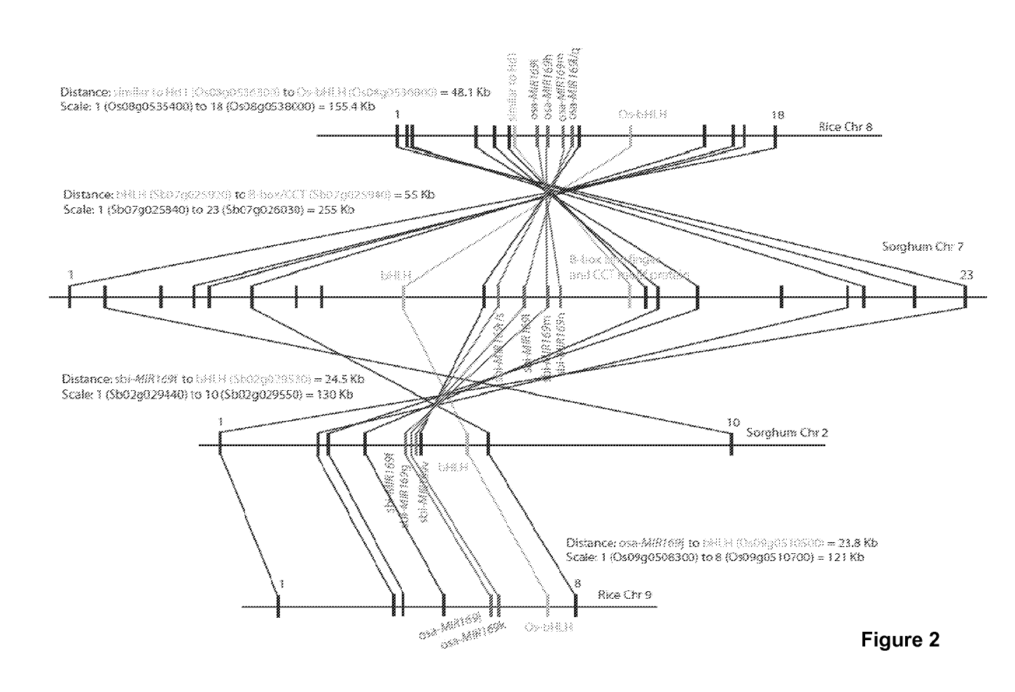

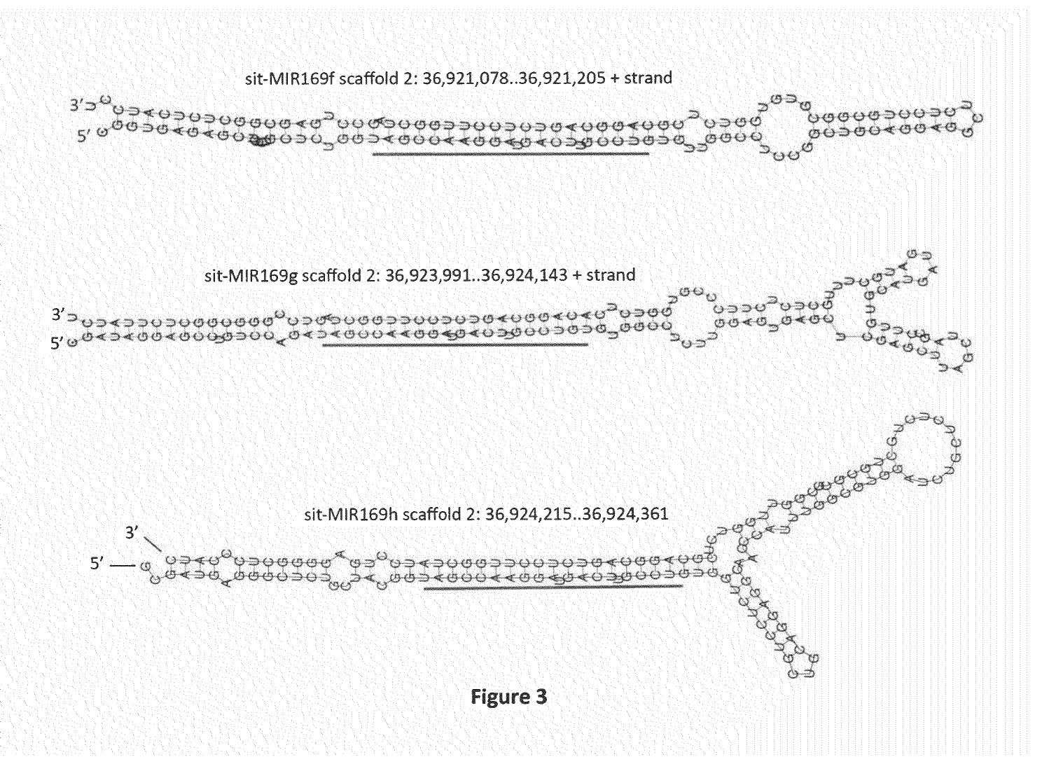

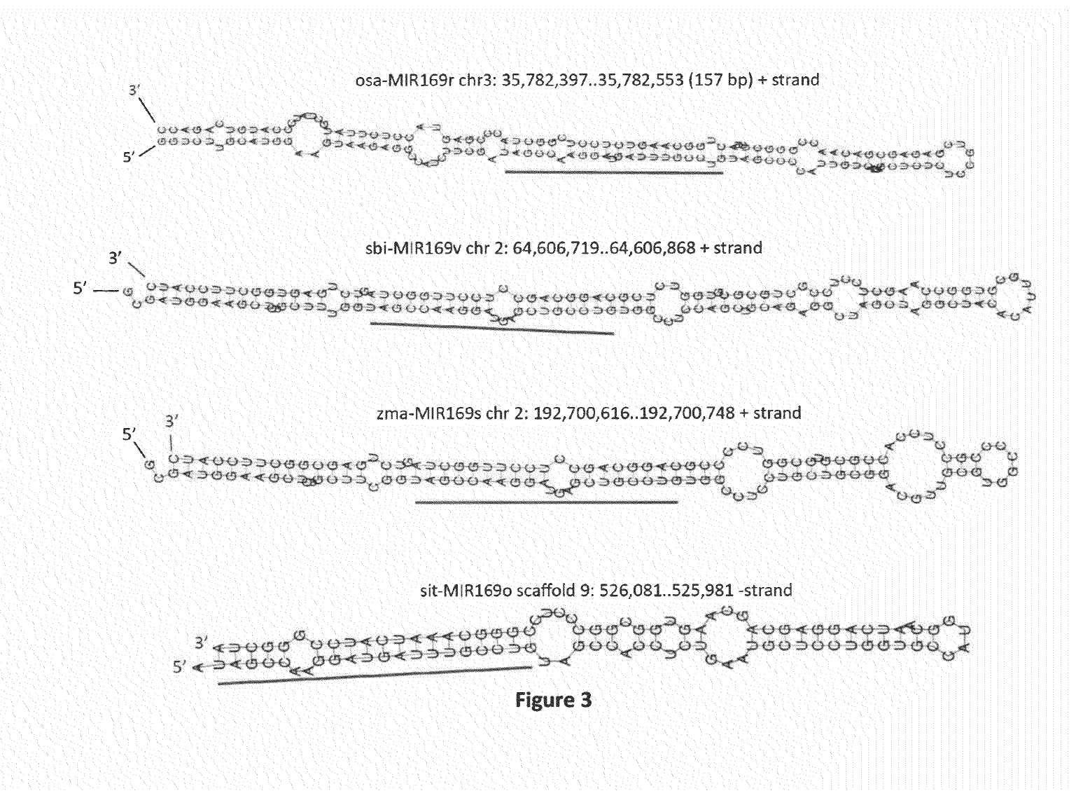

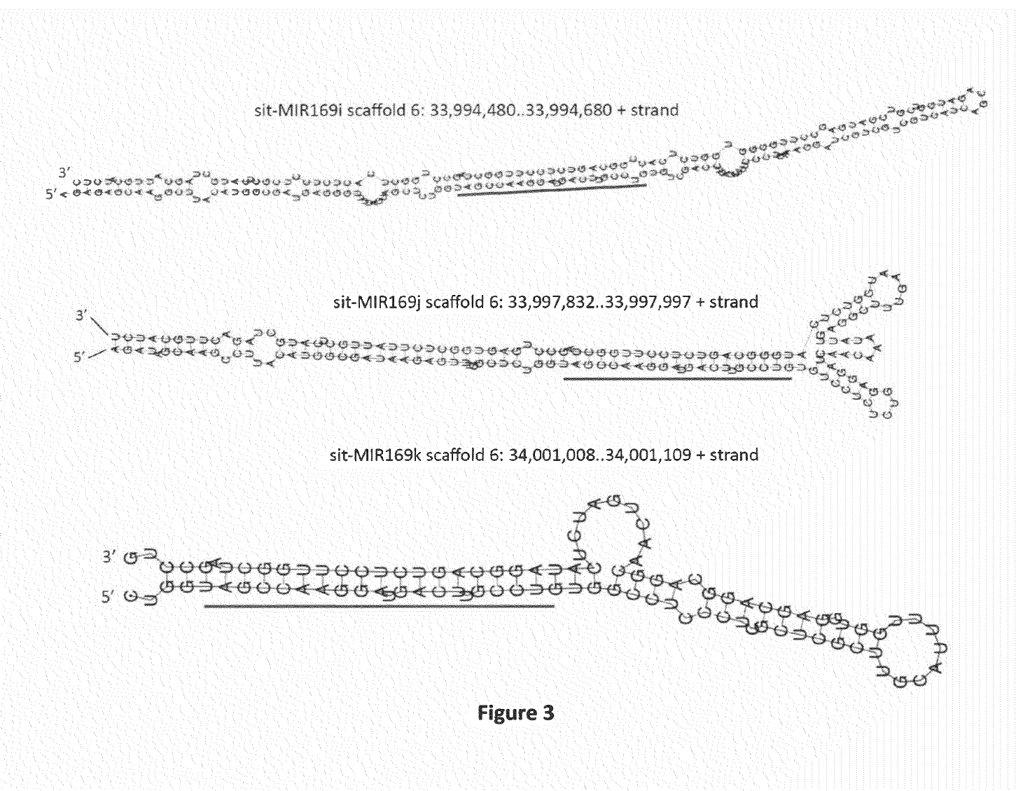

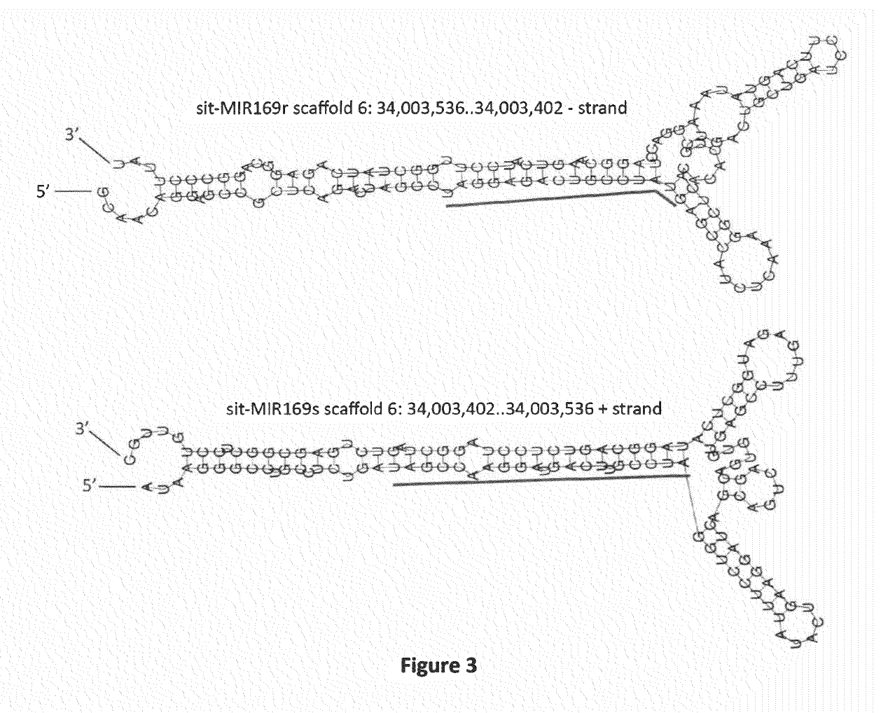

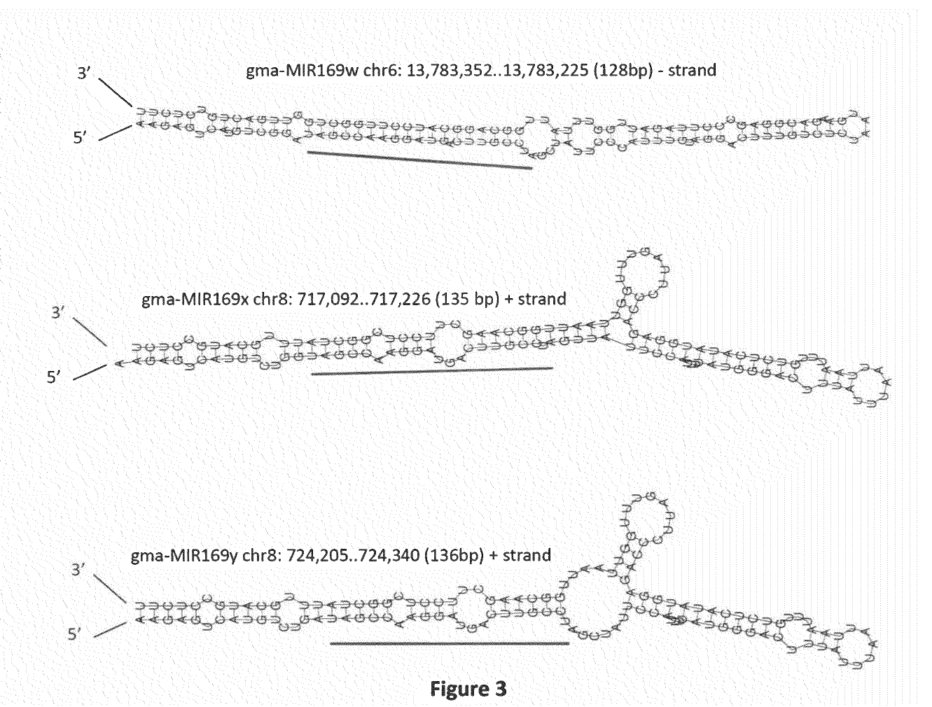

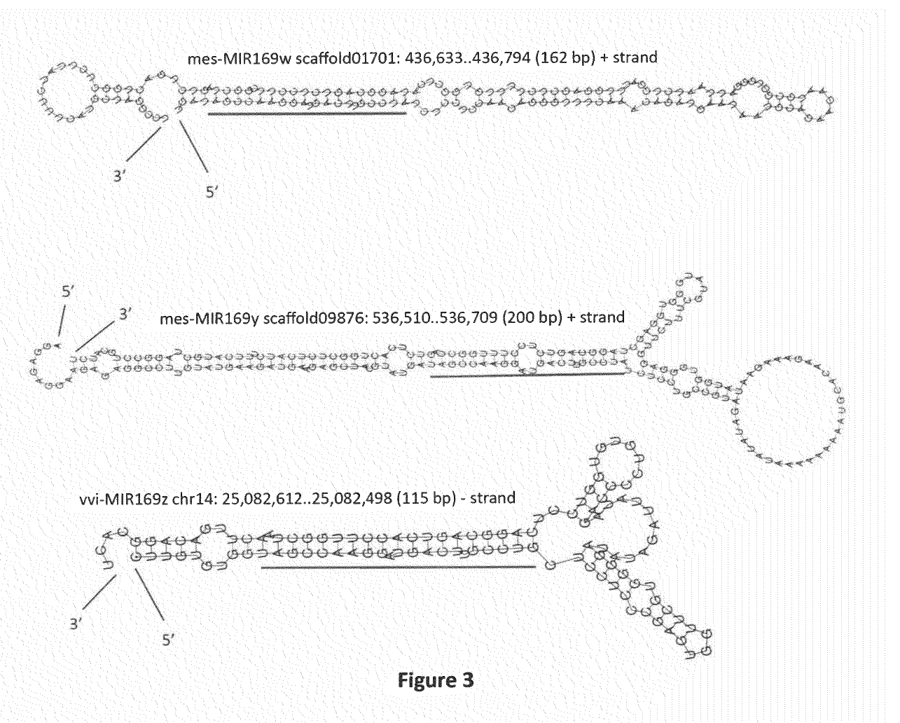

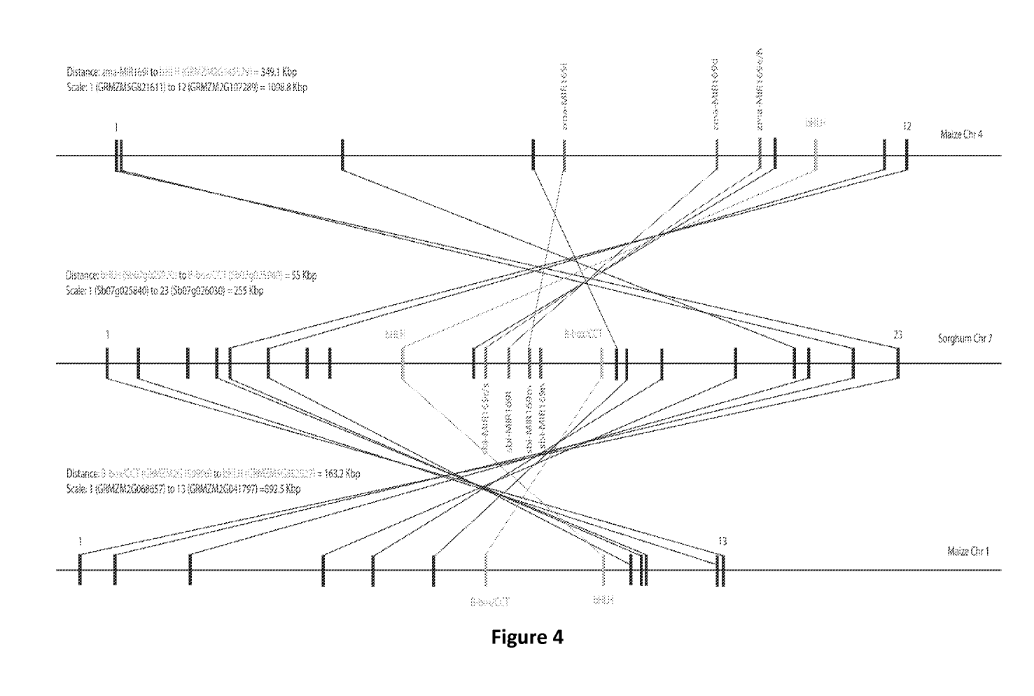

We first analyzed the region containing the MIR169 cluster on sorghum chr7 because it had the highest number of gene copies. The alignment of sorghum genes flanking MIR169 copies to the rice genome permitted the identification of a collinear region on rice chr8 also containing a cluster of MIR169 gene copies (FIG. 2). Interestingly, the cluster on rice chr8 was composed of five MIR169 gene copies whereas the orthologous cluster on sorghum chr7 contained only three annotated MIR169 gene copies. Further investigation based on reciprocal Blastn analysis revealed that osa-MIR1691 and osa-MIR169q are orthologous to a region on sorghum chr7, where there was no previous annotation of MIR169 genes. Indeed, by taking the sorghum DNA segment highly similar to osa-MIR1691 and osa-MIR169q and subjecting it to an RNA folding program (RNAfold: http://rna.tbi.univie.ac.at/cgi-bin/RNAfold.cgi) in order to identify hairpinlike structures characteristic of microRNA precursors, we were able to discover two new MIR169 gene copies in sorghum that we named sbi-MIR169r and sbi-MIR169s, respectively (FIG. 2 and FIG. 3). Independent support for the new annotation of sbi-MIR169r and sbi-MIR169s was achieved through orthologous alignment of a third species, maize, through zma-MIR169e and zma-MIR169h gene copies (FIG. 4).

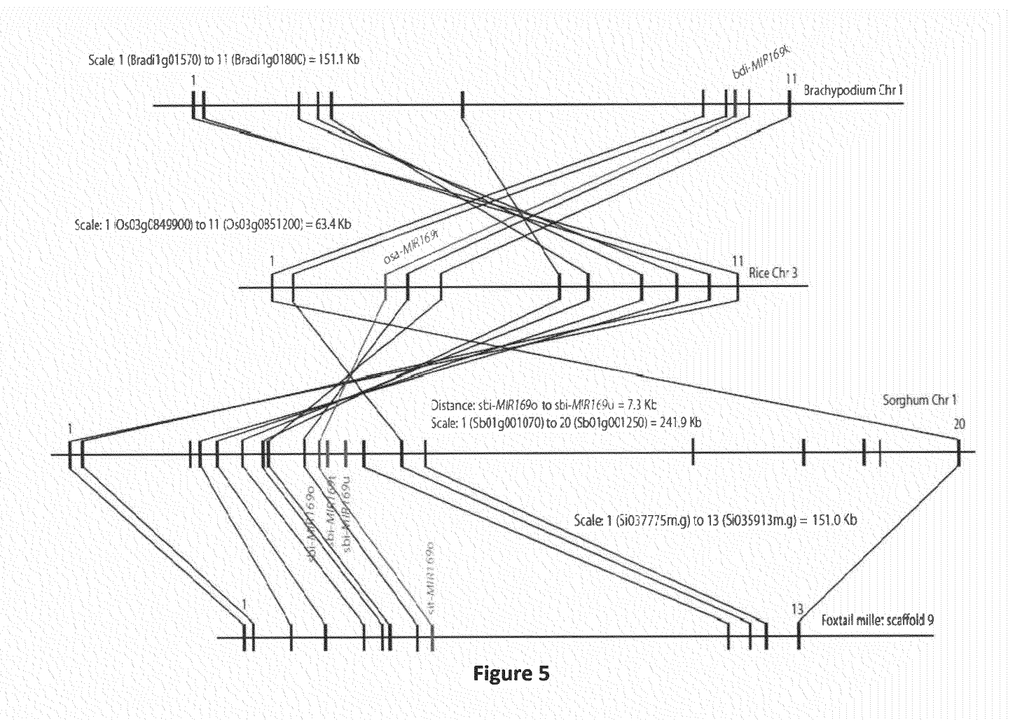

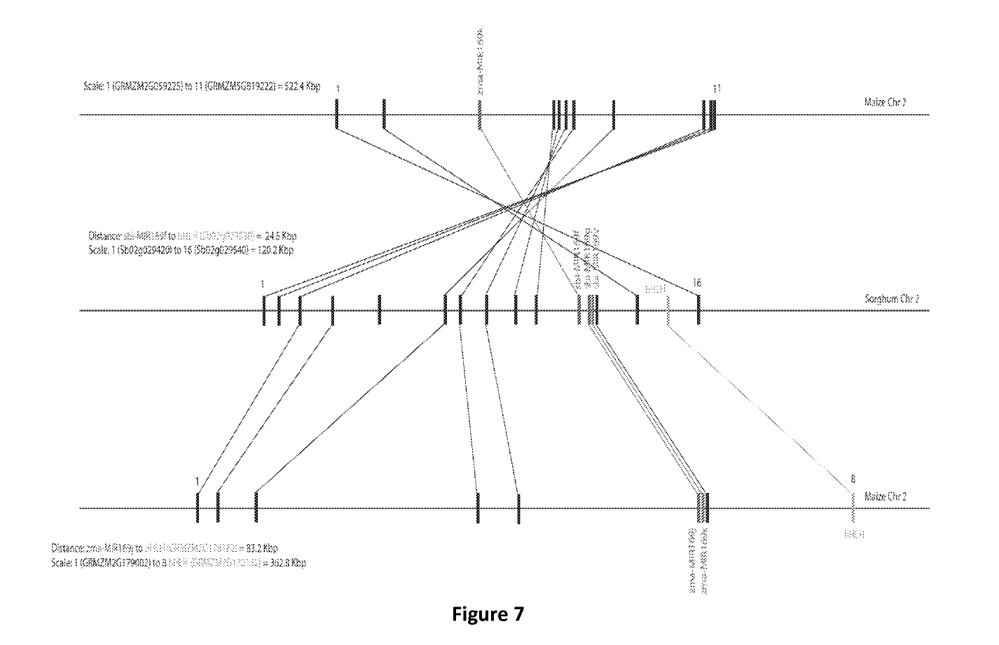

To identify additional MIR169 gene copies in sorghum that might have arisen by tandem duplication, we took each of the annotated MIR169 genes and performed Blastn analysis against the sorghum genome to search for new copies located in close proximity to any of the previously annotated ones. Such analysis identified two new MIR169 copies on sorghum chr1 when sbi-MIR1690 was used as query that we named sbi-MIR169t and sbi-MIR169u, respectively (FIG. 3). Thus, sbi-MIR1690 together with sbi-MIR169t and sbi-MIR169u constituted a new MIR169 cluster of the sorghum genome (Table 1). The segment containing the newly identified MIR169 cluster on sorghum chr1 was collinear with an orthologous segment of rice chr3 (FIG. 5), although no MIR169 gene had previously been found in this region. By performing reciprocal Blastn analysis with sbi-MIR1690 against the rice genome we could identify the corresponding orthologous MIR169 copy on rice chr3 that we named osa-MIR169r (FIGS. 3 and 5). Furthermore, osa-MIR169r is contained within a segment that is collinear with an orthologous region of chr1 of a fourth species, Brachypodium, corresponding to bdi-MIR169k (FIG. 5). Comparison between sorghum and maize revealed that the MIR169 cluster on sorghum chr1 is collinear with a segment on maize chr1 that contains zma-MIR1691 (FIG. 6). Indeed, sbi-MIR169u and zma-MIR1691 are also orthologous gene copies. Finally, when the cluster on sorghum chr2 containing sbi-MIR169f and sbi-MIR169g was analyzed, collinearity with the segment on sorghum chr7 containing the sbi-MIR169r/s and sbi-MIR1691-n cluster revealed the existence of an additional MIR169 copy on sorghum chr2 that we named sbi-MIR169v (FIG. 2; FIG. 3; Table 1). Furthermore, the sbi-MIR169f/g/v cluster is syntenic with a region on maize chr7 containing zma-MIR169k and its homoeologous region on maize chr2 containing zma-MIR169j and the newly identified zma-MIR169s gene copy (FIG. 3 and FIG. 7; Table 1).

In summary, by aligning sorghum chromosomal segments containing MIR169 clusters with orthologous regions of Brachypodium, rice, and maize we were able to identify 5 additional MIR169 copies in sorghum and an additional copy in rice and maize, respectively.

### New MIR169 Clusters in the Recently Sequenced Foxtail Millet Genome

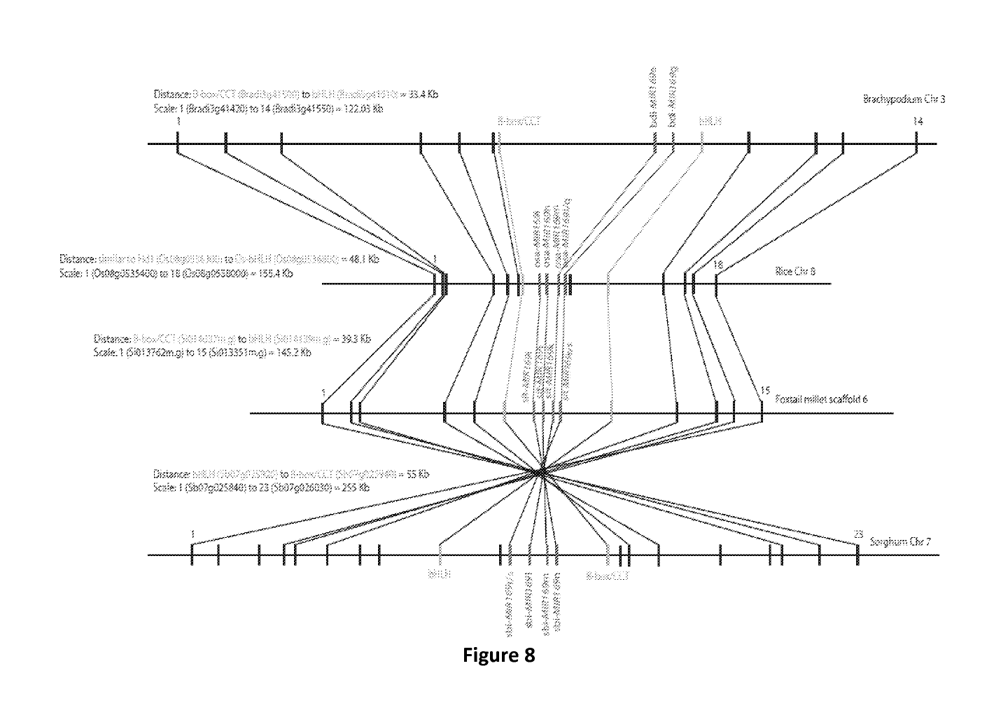

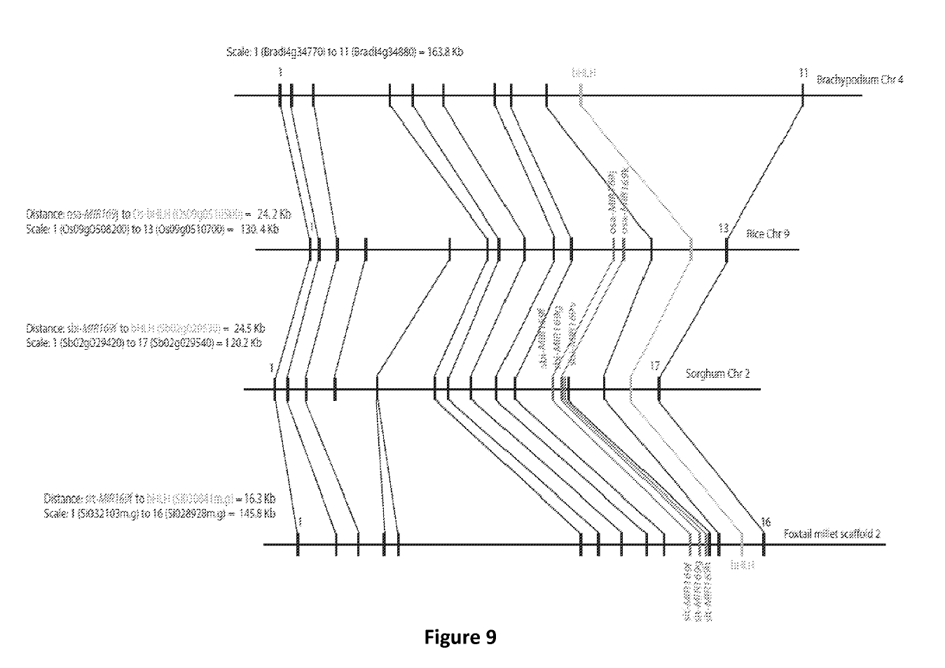

The recent release of the complete reference genome sequence for foxtail millet (Setaria italica) (Bennetzen, et al. 2012; Zhang, et al. 2012) greatly enhances comparative genomics analysis within the Poaceae, with genome sequences available from five species. Foxtail millet provided us with additional information to study syntenic relationships with sorghum because they split from each other about 26 million years (myr) ago (Zhang, et al. 2012). Indeed, 19 collinear blocks were found between foxtail millet and sorghum, which comprised about 72% of the foxtail millet genome (Zhang, et al. 2012). Consequently, we could use sorghum to identify and predict MIR169 gene copies in the foxtail millet genome. We identified and predicted MIR169 copies in foxtail millet, collinear with sorghum MIR169 copies, arranged in clusters on chr1, chr2, and chr7, respectively. The sorghum MIR169 cluster on chr1 was collinear with a segment on chr9 of foxtail millet, from which sit-MIR1690 was identified as the ortholog of sbi-MIR1690 (FIG. 5; FIG. 3; Table 1). The sorghum MIR169 copies arranged in cluster on chr7 were collinear with a segment on chr6 from foxtail millet that harbored the newly identified orthologous MIR169 copies sit-MIR169i, sit-MIR169j, sit-MIR169k, sit-MIR169r, and sit-MIR169s (FIG. 8; FIG. 3; Table 1). Finally, tandem sorghum MIR169 copies on chr2 were collinear with a segment on foxtail millet chr2 that contained the three newly predicted MIR169 copies sit-MIR169f, sit-MIR169g and sit-MIR169h (FIG. 9; FIG. 3; Table 1).

In summary, we used sorghum as a reference genome to identify and predict nine MIR169 gene copies that were collinear with foxtail millet. The prediction of MIR169 genes in the foxtail millet will greatly facilitate their experimental validation through the sequencing of small RNAs from different tissues and developmental stages.

### Gain and Losses of MIR169 Gene Copies During Grass Evolution

To determine expansion and contraction of the MIR169 gene clusters, we aligned collinear chromosomal segments of diploid Brachypodium, rice and foxtail millet, and the two homoeologous regions of allotetraploid maize. Based on nucleotide substitution rates, the cluster of MIR169 copies on sorghum chr7 was likely preserved from an ancestral grass chromosome and comprised five MIR169 gene copies, from which three of them were deleted in Brachypodium after the split of Brachypodium from the ancestor of rice, foxtail millet, and sorghum (FIGS. 8 and 10A and 10B). The number of MIR169 genes (five copies per cluster) was unchanged in rice, sorghum, and foxtail millet, whereas in maize four copies were retained on orthologous homoeologous region on chr4 but none on the homoeologous region on chr1 (FIG. 4 and FIG. 10A). Although the MIR169 copies were deleted from maize chr1, the flanking genes remained intact.

In the case of the MIR169 cluster on sorghum chr2, its evolution can be explained according to two models (FIG. 10A). In the first one, the ancestor of the grasses had two MIR169 copies and they were conserved before the split of Brachypodium and rice, with Brachypodium losing these two MIR169 copies were rice maintained them. An additional copy was gained in the common ancestor of foxtail millet, sorghum and maize, giving rise to a cluster with three MIR169 gene copies. Phylogenetic analysis suggested that the new copy in the ancestor of foxtail millet, sorghum, and maize was the ancestral copy that gave rise to sit-MIR169h, sbi-MIR169v and zma-MIR169s, respectively (FIG. 10C). We estimated that the time at which this copy arose in the progenitor of foxtail millet, sorghum and maize was about 41.1 mya (see methods section for estimation of time of duplication). Alternatively, the common ancestor of the grasses could have had three MIR169 gene copies and one copy was lost in the common ancestor of Brachypodium and rice, with a subsequent loss of two additional MIR169 gene copies in Brachypodium relative to rice (FIG. 10A).

Regarding the cluster of MIR169 copies on sorghum chr1, we favor a model where the ancestor of the grasses had a single MIR169 copy because Brachypodium, rice and foxtail millet all have a single MIR169 copy (FIG. 10D). Thus, the additional two MIR169 copies present in the sorghum cluster could have arisen via duplication events. Phylogenetic analysis suggested that the ancestral copy in the cluster was sbi-MIR169o, from which sbi-MIR169t subsequently duplicated 8.5 mya (see methods) (FIG. 10D). Thus, sbi-MIR169t was acquired specifically in the sorghum lineage. Since sbi-MIR169u and zma-MIR1691 are highly related but distantly related from sbi-MIR1690 and sbi-MIR169t (FIG. 10D), we postulate that the ancestral copy of sbi-MIR169u and zma-MIR1691 was inserted next to the other MIR169 gene copies in the progenitor of sorghum and maize. In the maize lineage, diploidization after allotetraploidization led to the deletion of the corresponding orthologous MIR169 copy from the homoeologous segment on chr5, whereas the flanking genes remained conserved (FIG. 6).

In summary, differences in MIR169 copy number between clusters from Brachypodium, rice, foxtail millet, sorghum and maize arose by duplication of ancestral MIR169 genes that were retained or lost during grass evolution. Overall, sorghum gained eight MIR169 copies relative to Brachypodium, three copies relative to rice, two copies relative to foxtail millet and three copies relative to maize. Polymorphisms in chromosomal inversions containing MIR169 clusters Through the analysis of three chromosomal regions in sorghum containing MIR169 clusters and their alignment with the genomes of Brachypodium, rice, foxtail millet, and maize we were able to identify four chromosomal inversions in total, one in rice chr3 containing osa-MIR169r (FIG. 5), a second on sorghum chr7 containing sbi-MIR169r, sbi-MIR169s, sbi-MIR1691, sbi-MIR169m and sbi-MIR169n (FIG. 2), a third on maize chr1 containing zma-MIR1691 (FIG. 6) and the fourth on maize chr7 containing zma-MIR169k (FIG. 7), respectively. The inversion on rice chr3 was absent from the corresponding collinear regions on Brachypodium chr1, sorghum chr1 and foxtail millet chr9 (FIG. 5), indicating that the inversion happened after the split of rice from the common ancestor of sorghum and foxtail millet. The region on sorghum chr1 containing sbi-MIR169o, sbi-MIR169t and sbi-MIR169u that was collinear with the inverted segment on rice chr3 was also collinear with an inverted segment on the homoeologous region of maize chr1 containing zma-MIR1691 (FIG. 6). However, the inversion did not occur on the homoeologous region on maize chr5, indicating that the inversion occurred after the allotetraploidization event that took place in maize. The inversion on sorghum chr7 containing sbi-MIR169r, sbi-MIR169s, sbi-MIR1691, sbi-MIR169m and sbi-MIR169n cluster only occurred in this species (FIG. 4 and FIG. 8), suggesting that it took place after the split of sorghum from the common ancestor of sorghum and maize. The MIR169 cluster on sorghum chr2 was collinear with an inverted region on maize chr7 containing zma-MIR169k (FIG. 7). The homoeologous region on chr2 did not exhibit the inversion, suggesting that it took place after the allotetraploidization event that occurred in maize.

In summary, four inversions containing MIR169 copies were found in total, one in rice, one in sorghum and two in maize. These inversions were lineage specific as none of them was present in a collinear region in the genome of a second grass species, indicating that these inversions happened after the species were formed.

### Validation of Newly Identified MIR169 Gene Copies in Sorghum and Maize

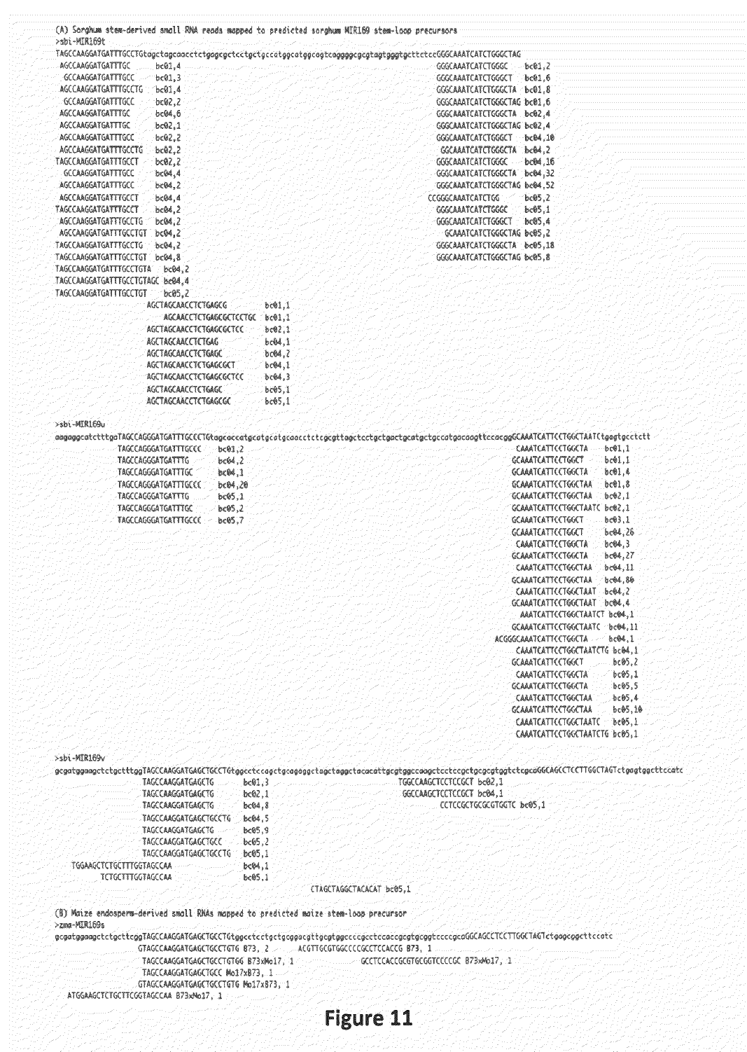

In order to experimentally validate the new MIR169 gene copies found in sorghum through our syntenic analysis among grasses, we mapped previously sequenced small RNAs from sorghum stems (Calvino, et al. 2011) to the newly predicted MIR169t/u/v/r/s hairpins. Similarly, to validate the newly described zma-MIR169s gene copy in maize, we constructed small RNA libraries from endosperm tissue belonging to cultivars B73, Mo17 and their reciprocal crosses (Table 2). Maize endosperm-derived small RNAs were then mapped to the new MIR169s hairpin annotated in this study. We could effectively map small RNA reads to the stem-loop sequences of all five predicted microRNA169 in sorghum (with respect of sbi-MIR169r/s see next section). In the case of sbi-MIR169t and sbi-MIR169u, the most abundant small RNA reads were derived from the miR169* sequence (FIG. 11) although small RNAs derived from the canonical miR169 sequence were also found but in less abundance. The experimental validation of sbi-MIR169v was supported with mapping of small RNAs to the corresponding predicted mature miR169v sequence (FIG. 11). Regarding the experimental validation of the predicted zma-MIR169s copy in maize, we were able to detect small RNA reads derived from miR169s although their abundance was very low (Supplemental FIG. 5).

TABLE 2
Deep sequencing statistics of maize endosperm-derived small RNAs

| | | # Sequences With Perfect | |
|---|---|---|---|
| Library | # Raw Sequences | Match to B73 Genome | % |
| B73 | 14,371,575 | 3,805,955 | 26.48 |
| Mo17 | 16,207,393 | 7,688,661 | 47.44 |
| B73 × Mo17 | 13,051,982 | 5,985,649 | 45.86 |
| Mo17 × B73 | 19,924,315 | 6,514,306 | 32.7 |

Antisense microRNA169 Gene Pairs Generate Small RNAs that Target Different Set of Genes

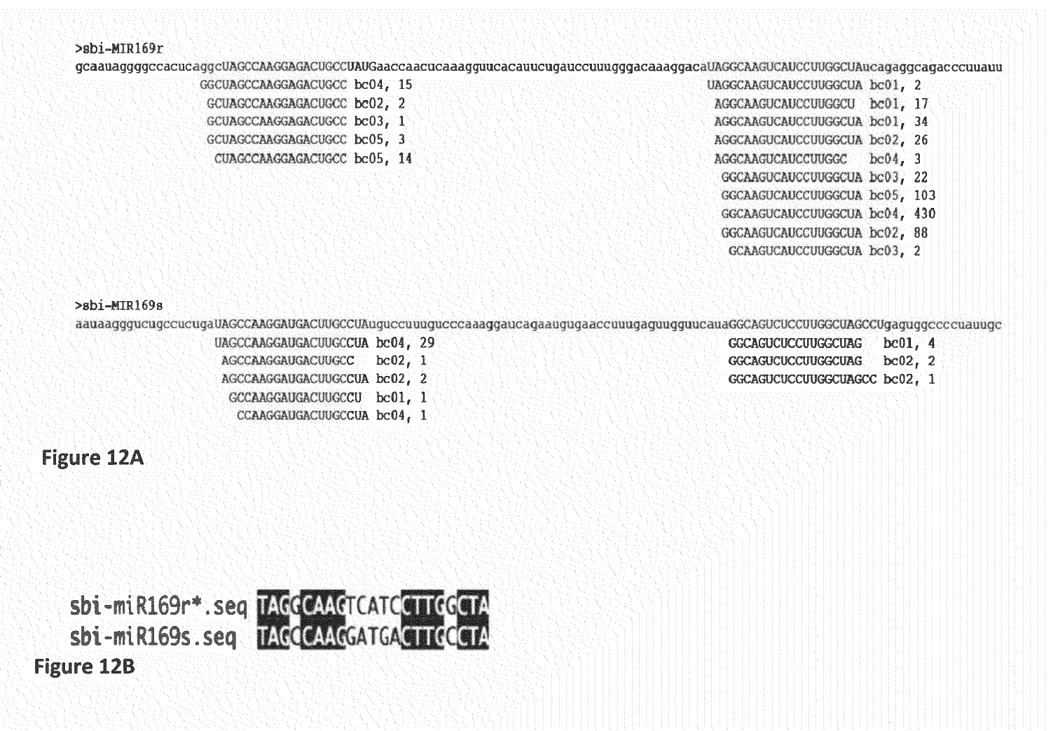

In rice, osa-MIR1691 and osa-MIR169q were annotated as antisense microRNAs and small RNA reads derived from both strands were identified (Xue, et al. 2009). In sorghum, sbi-MIR169r, and sbi-MIR169s are collinear with osa-MIR1691/q (FIGS. 2 and 8) and are antisense microRNAs as well (FIGS. 3 and 10A). Despite the lack of EST evidence for sbi-MIR169r and sbi-MIR169s annotation, our previously generated small RNA library from sorghum stem tissue (Calvino, et al. 2011) supported the transcription from both strands based on small RNA reads mapped to both sbi-MIR169r and sbi-MIR169s, respectively (FIG. 12A). Similarly, EST evidence supported the transcription from opposite strands in the microRNA antisense pair zma-MIR169e/h (ESTs ZM_BFb0354L14.r and ZM_BFb0294A24.f, respectively). Because small RNAs derived from zma-MIR169e/h had not been previously reported (miRBase database: release 19, August 2012), we used the SOLiD system to sequence small RNAs from endosperm tissue derived from B73 and Mo17 cultivars and their reciprocal crosses, however we could not detect small RNA reads derived from them, at least in endosperm tissue. Thus, antisense microRNAs from MIR169 gene copies are being actively produced in rice and sorghum, and possibly in maize.

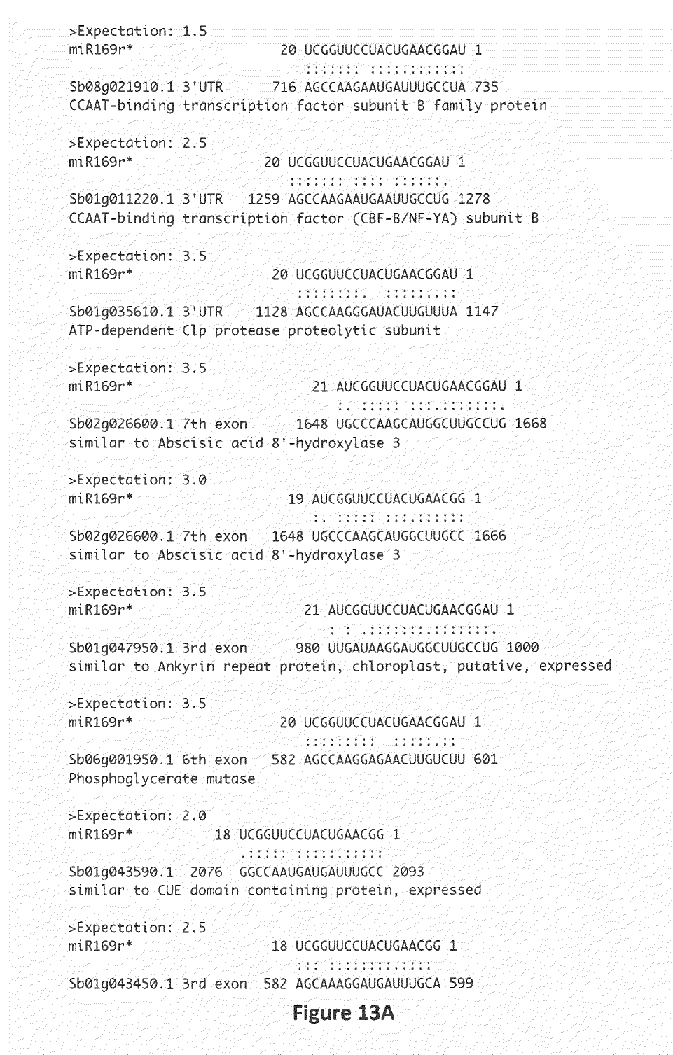

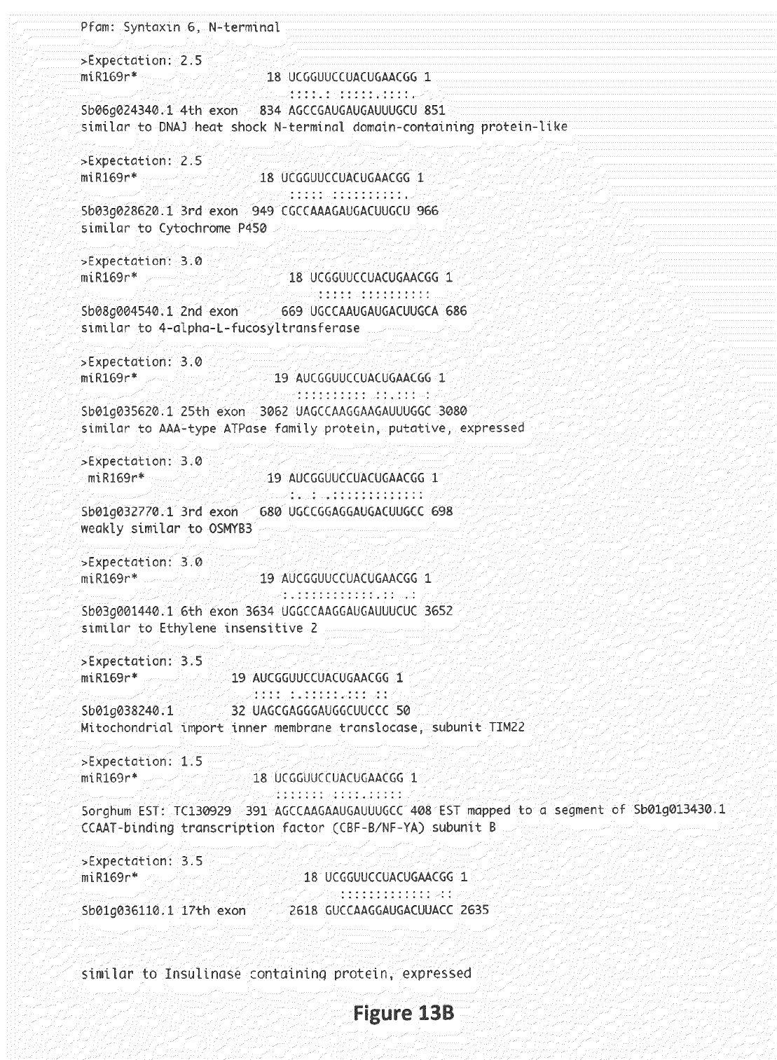

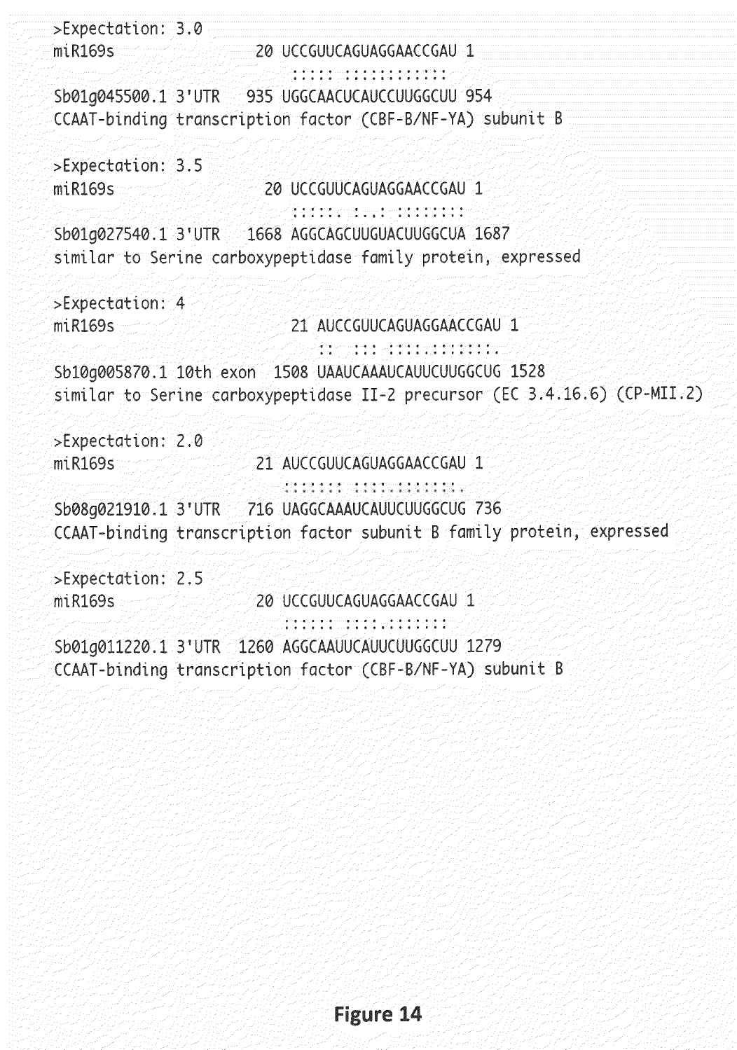

With respect to sbi-MIR169r/s antisense gene pair, we found that the small RNA reads mapped to sbi-MIR169r were predominantly associated with the miR169r* sequence (FIG. 12A). The mature miRNA sequences for sbi-miR169r* and sbi-miR169s differed from each other in 7 nucleotides (FIG. 12B). Moreover, they would have different set of genes as targets based on their sequences (FIGS. 13 and 14). Moreover, the assumption that also microRNA* have functional roles was recently described (Meng, et al. 2011; Yang, et al. 2011).

Linkage of MIR169 Gene Copies with Flowering and Plant Height Genes

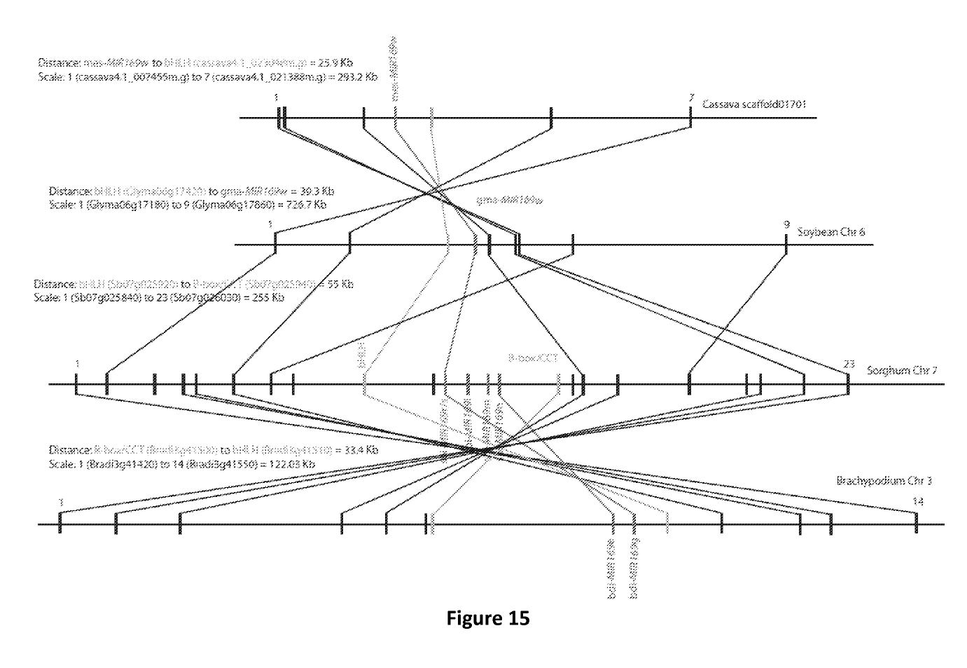

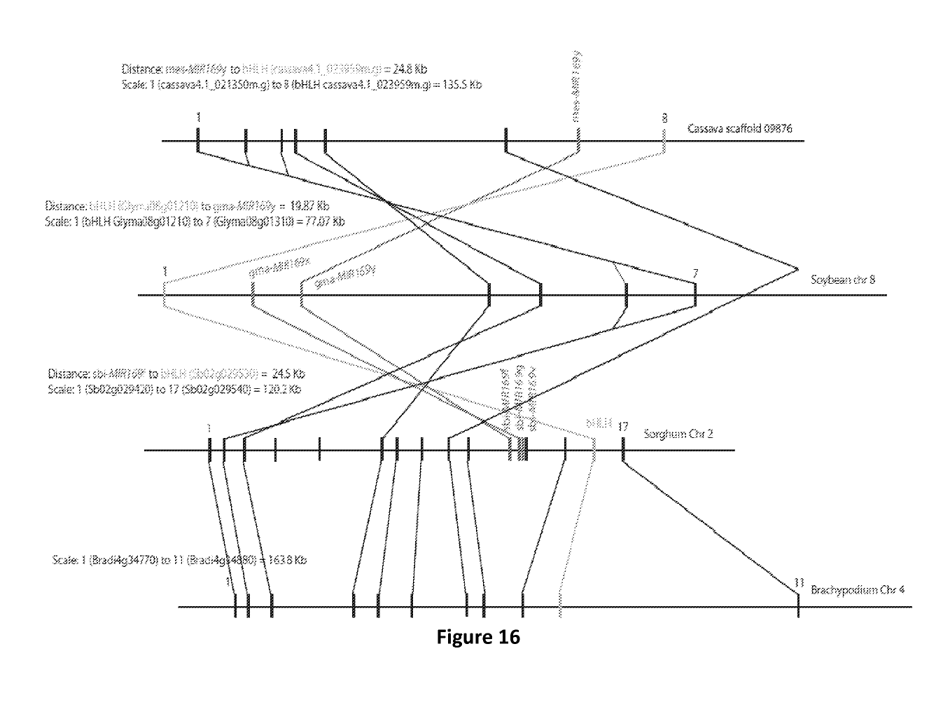

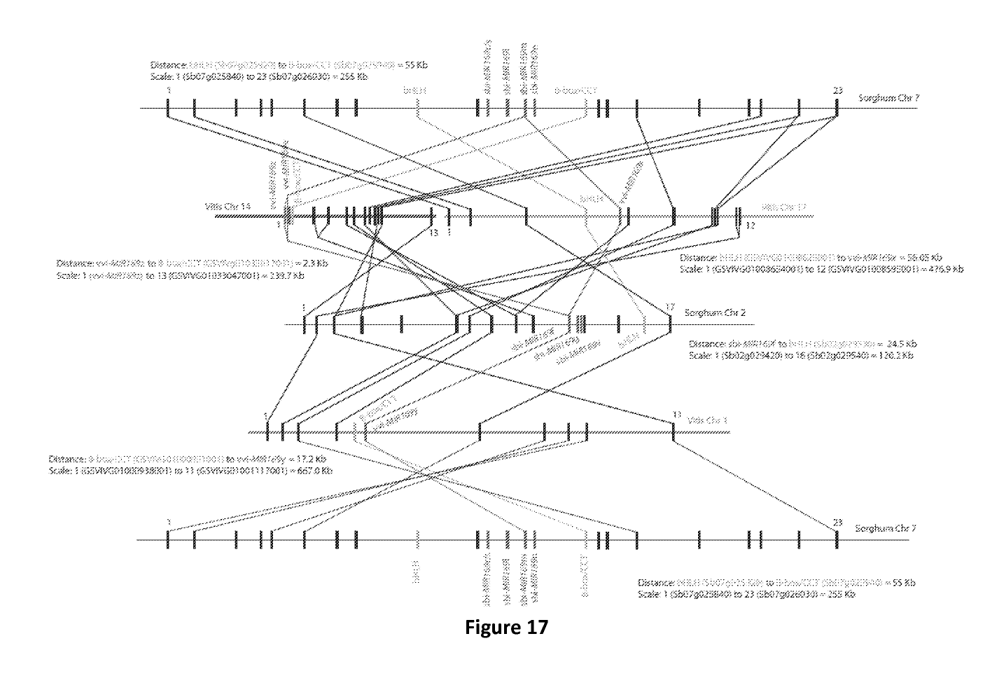

Based on the alignment of collinear regions containing MIR169 genes located on sorghum chr2 and chr7, we noticed a tight linkage of MIR169 copies with two genes encoding a bHLH protein, and a B-box zinc finger and CCT-motif protein that were similar to Arabidopsis bHLH137 and CONSTANS-LIKE 14 proteins (FIGS. 2, 8 and 9 and FIGS. 4 and 7). The Arabidopsis bHLH137 and COL14 genes were described to have a role in gibberellin signaling (mutations in genes involved in gibberellin signaling and/or perception affects plant height (Fernandez, et al. 2009)) and flowering time, respectively (Griffiths, et al. 2003; Wenkel, et al. 2006; Zentella, et al. 2007). The physical linkage of MIR169 gene copies to bHLH and COL genes (or any of the two) was present in all of the five grasses examined. We hypothesized that the physical association of MIR169 to either of these flowering and/or plant height genes could be of relevance because of previously reported trade-offs in sorghum between sugar content in stems and plant height and flowering time, respectively (Murray, et al. 2008). For breeding purposes, the introgression of a particular gene/phenotype from a specific cultivar into another would consequently also bring in the neighboring gene, a process known as linkage drag. Furthermore, linkage drag between MIR169 copies and the bHLH and COL genes could also be of ecological importance because a single chromosomal segment comprises genes involved in drought tolerance, sugar accumulation, and flowering. If this is case, linkage of MIR169 copies to either bHLH or COL genes could have been preserved even after the monocotyledoneous diversification. Indeed, we were able to find collinearity between chromosomal segments containing MIR169 and bHLH genes from Brachypodium, sorghum, soybean, and cassava (FIG. 15). Moreover, we found that the physical linkage between MIR169 and the bHLH gene on sorghum chr7 was retained in collinear regions of soybean chr6 and cassava scaffold 01701, respectively (FIG. 15). Similarly, the physical/genetic association of MIR169 with the bHLH gene from sorghum chr2 was retained in the corresponding collinear regions from soybean chr8 and cassava scaffold 09876 (FIG. 16). Interestingly, the linkage between MIR169 and the COL gene that was present in Brachypodium chr3 and sorghum chr7 was broken in the corresponding collinear regions of soybean chr6 and cassava scaffold 01701 (FIG. 15). We then compared the two MIR169 clusters from sorghum chr2 and chr7 to the grapevine genome because grapevine and sorghum are more closely related than sorghum to soybean and cassava, respectively. Our comparison revealed a two-to-three relationship between sorghum and grapevine (FIG. 17), and this is consistent with the palaeo-hexaploidy event that took place in the grapevine genome (Jaillon, et al. 2007). The physical/genetic linkage of MIR169 copies with the COL gene on sorghum chr7 was preserved in two out of the three homoeologous chromosomal segments in grapevine on chr1 and chr14, whereas the third homoeologous segment on chr17 retained the close association of MIR169 with the bHLH gene.

The finding of micro-synteny conservation between monocots and dicots species in chromosomal segments containing MIR169 gene copies together with bHLH and COL genes is remarkable because the estimated time of divergence between monocots and dicots is about 130-240 million years ago (mya) (Jaillon, et al. 2007; Wolfe, et al. 1989). Such micro-synteny conservation permitted the discovery of new MIR169 gene copies in soybean (gma-MIR169w, gma-MIR169x and gma-MIR169y), cassava (mes-MIR169w and mes-MIR169y) and grapevine (vvi-MIR169z).

Subfunctionalization of the bHLH Gene in the MIR169 Cluster of Brachypodium

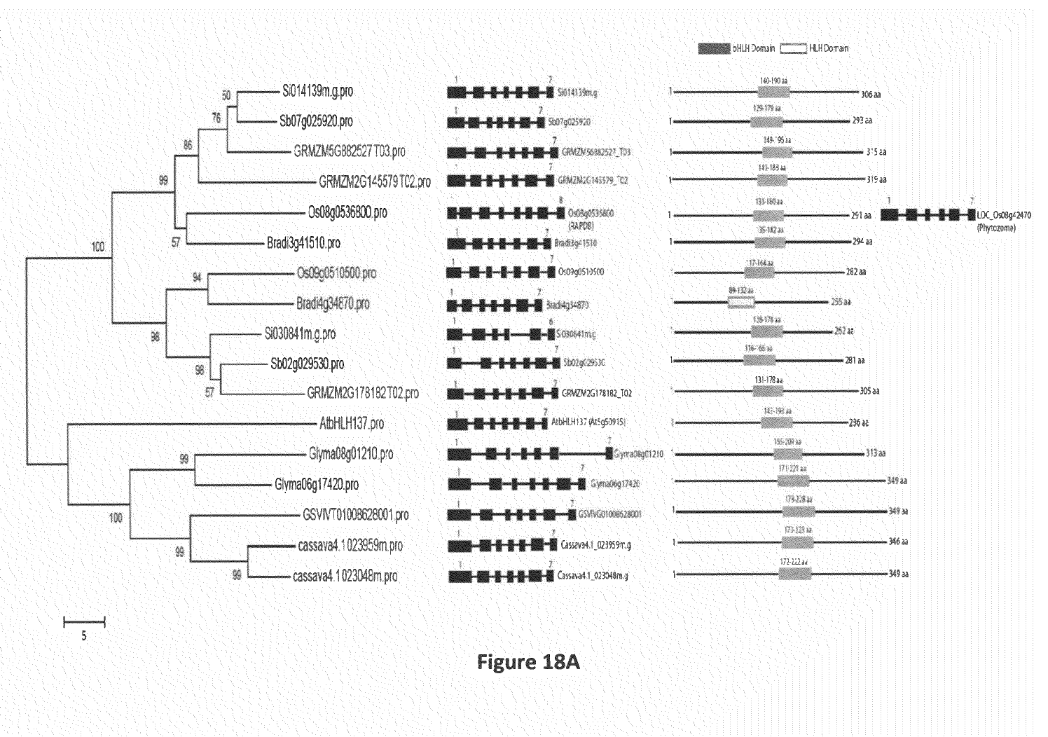

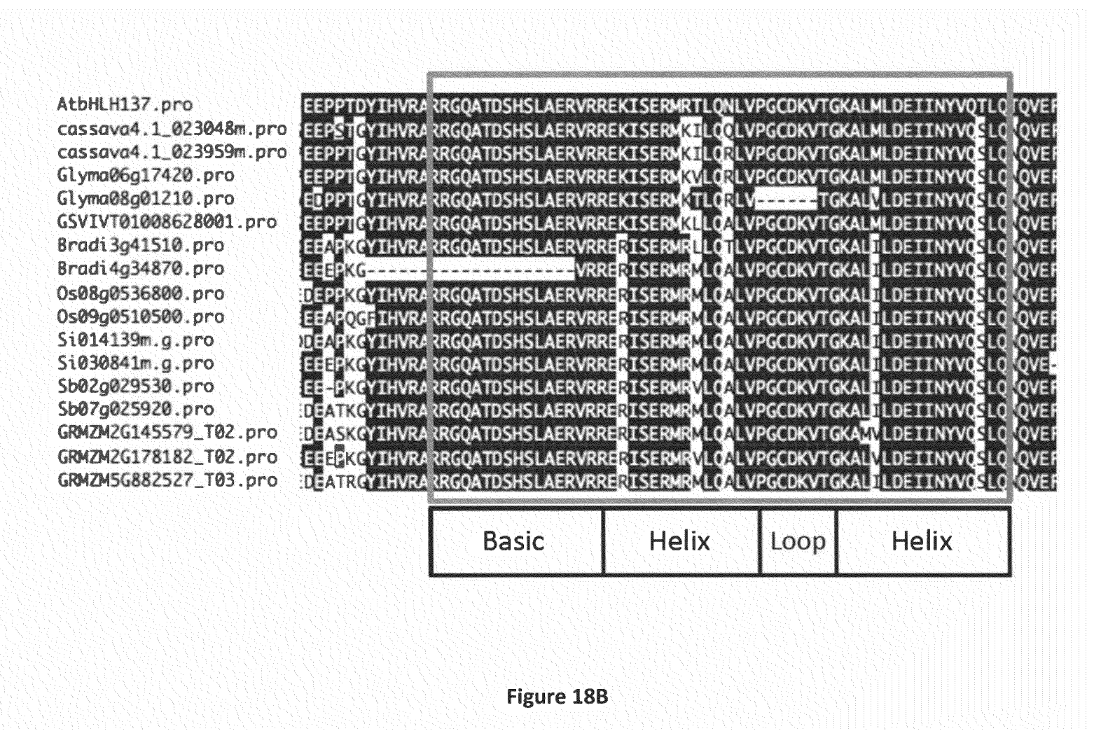

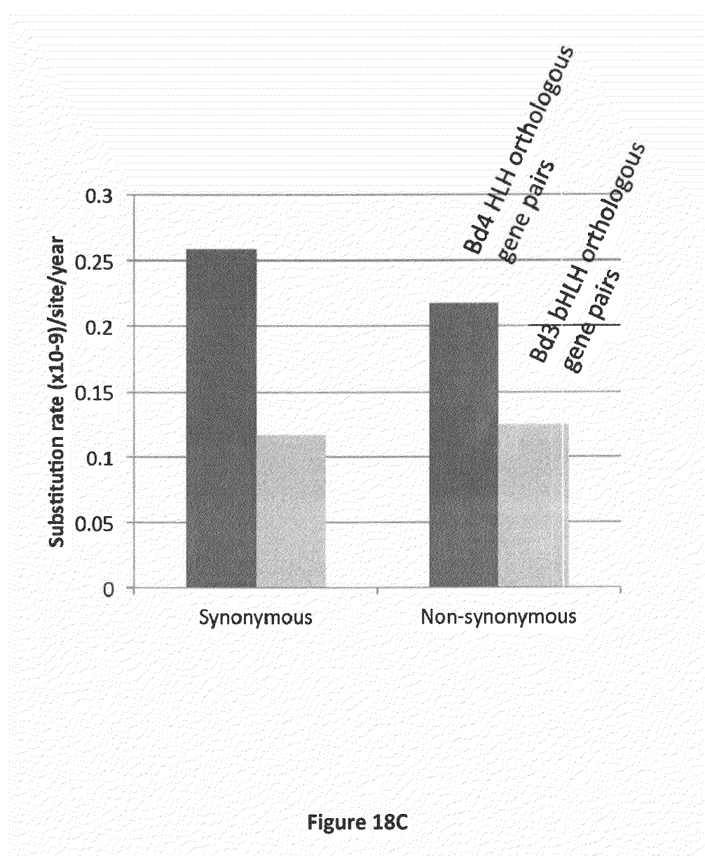

The microsynteny in chromosomal segments containing miR169 gene copies flanked by the bHLH gene among such distantly related species such as Brachypodium and cassava suggests that the linkage between miR169 and bHLH resulted from selection because of the divergence from a common ancestor about 130-240 mya. In support of this interpretation, the bHLH gene on Brachypodium chr4, where the miR169 cluster had been deleted, appeared to have undergone sub-functionalization. First, the bHLH copy on Brachypodium chr4 involved the loss of the basic domain, which is involved in DNA binding (Toledo-Ortiz 2003) and thus evolved into a HLH protein (FIGS. 18A and 18B). Because bHLH proteins act as homo- and/or heterodimers, where the basic domain of each bHLH protein bind DNA, HLH proteins homo- or heterodimerize and prevent the binding of the complex to DNA and thus becomes a negative regulator (Toledo-Ortiz 2003). Second, Brachypodium has a redundant intact orthologous copy on chr3, also a miR169 cluster next to it (FIG. 18). Third, the synonymous and non-synonymous substitution rate of the HLH orthologous gene pairs was higher than the synonymous and non-synonymous substitution rate in the bHLH orthologous gene pairs, respectively (FIG. 18C). Fourth, when we run a test for detecting adaptive evolution [calculated as the number of replacement mutations per replacement sites (dN) divided by the number of silent mutations per silent site (dS)] in the bHLH and HLH coding sequences, we found evidence on purifying selection on the HLH gene sequence (dN/dS ratio of −4.647).

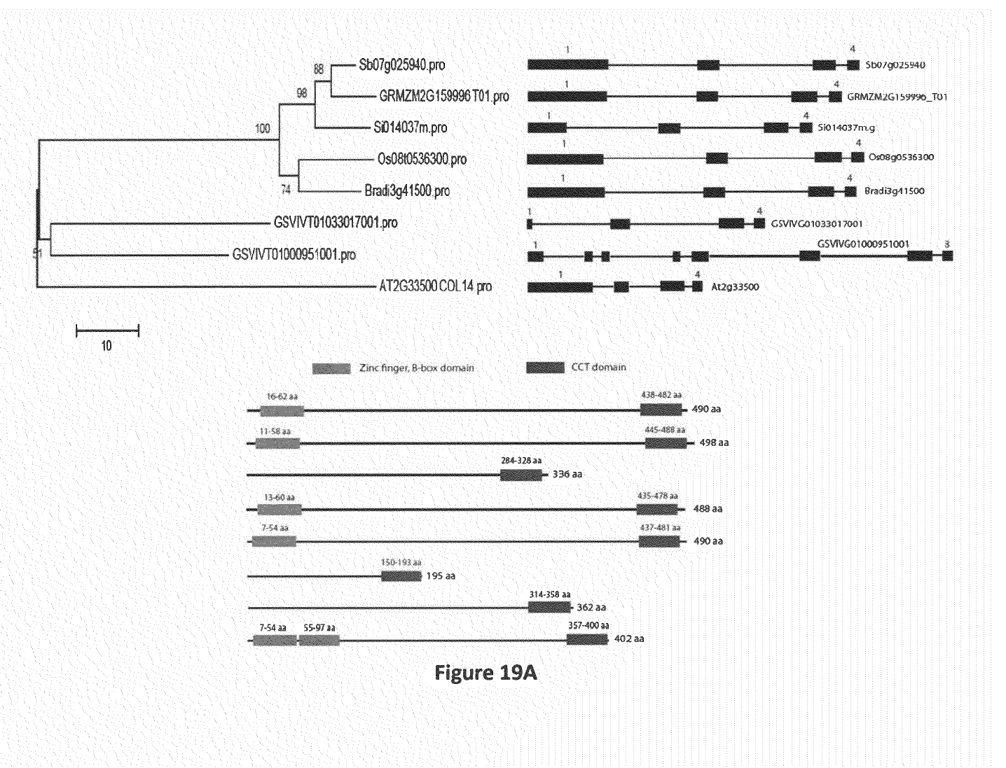

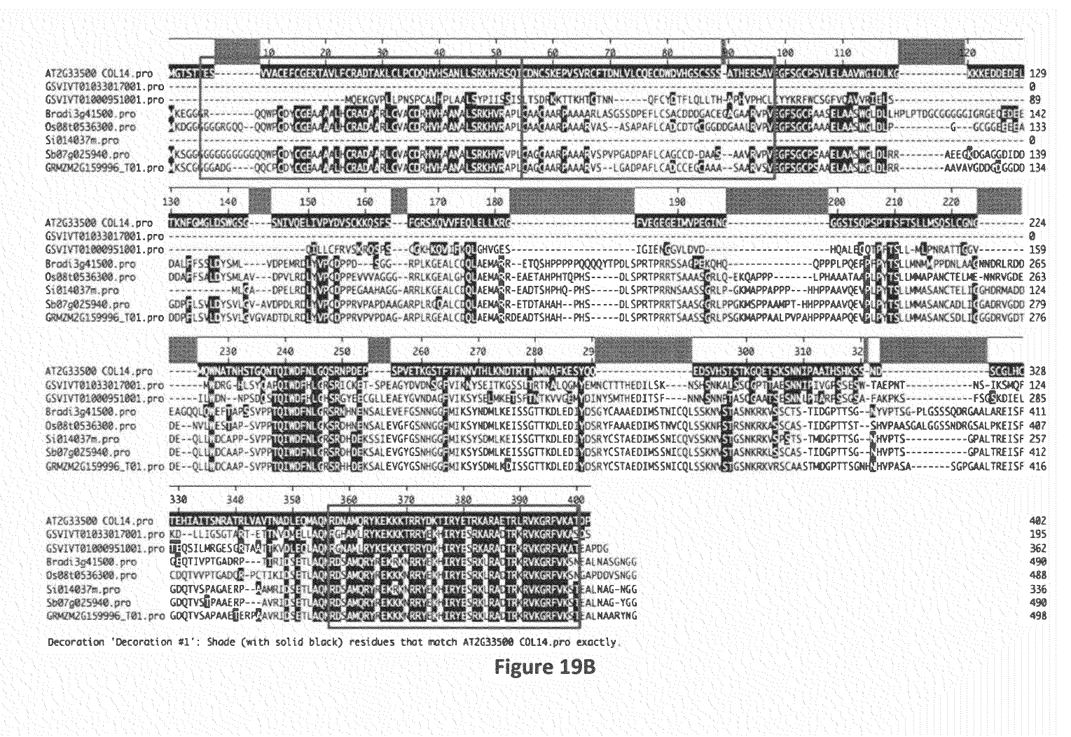

Conservation of synteny between sorghum and grapevine showed that the linkage between MIR169 gene copies and the COL gene was maintained in both species. Both COL genes in grapevine, on chr14 and on chr1, lost the B-box and zinc finger domain whereas the orthologous copy in sorghum retained it (FIGS. 19A and 19B). Similarly, foxtail millet COL protein lost the B-box and zinc finger domain whereas Brachypodium, rice, and maize retained it. The B-box and zinc finger domain are thought to mediate protein-protein interactions, whereas the CCT domain acts as a nuclear localization signal, with mutations in both domains causing flowering time phenotypes (Griffiths, et al. 2003; Valverde 2011; Wenkel, et al. 2006). Although the COL gene on grapevine chr14 has been recently identified as a candidate gene for a flowering QTL (Duchene, et al. 2012), the function of its corresponding orthologous copy on sorghum chr7 remains to be elucidated.

### Discussion

We describe the alignment of 25 chromosomal regions with orthologous gene pairs from eight different plant species. These regions contain a total of 48 MIR169 gene copies, from which 22 of them have been described and annotated here for the first time. The alignment of sorghum chromosomal regions containing MIR169 clusters to their corresponding orthologous regions from Brachypodium, rice, foxtail millet, and maize respectively, allows us not only to better understand the differential amplification of MIR169 gene copies during speciation, but also to identify new MIR169 gene copies not previously annotated in the rice, sorghum, and maize genomes. Our work highlights the usefulness of this approach in the discovery of microRNA gene copies in grass genomes and surprisingly also in dicotyledoneous genomes such as those from grapevine, soybean, and cassava. In addition, collinearity among grasses was used to predict and annotate MIR169 hairpin structures in the foxtail millet genome de-novo, from which no current microRNA annotation was available from the miRBase database (Release 19: August 2012). Our work suggests that synteny-based analysis should complement (whenever possible) homology-based searches of new microRNA gene copies in plant genomes.

Our analysis of MIR169 gene copies organized in clusters in the sorghum genome revealed that sorghum acquired eight MIR169 gene copies after Brachypodium split from a common ancestor, primarily due to gene losses (up to 5 MIR169 gene copies) in the Brachypodium lineage and new gene copies (up to 3) in the sorghum lineage (FIG. 6A). We propose that differences in MIR169 gene copy number between sorghum and Brachypodium is based on selective amplification in sorghum. Because diploidization of the maize genome resulted in the deletion of duplicated gene copies after allotetraploidization around 4.7 mya (Messing, et al. 2004; Swigonova, et al. 2004), also resulted in selective amplification in sorghum. Maize lost more than half, 9 out of 16 MIR169 gene copies, after allotetraploidization. Single gene losses in maize appear to be caused by short deletions that are predominantly in the 5 to 178 bp size range, with these deletions being about 2.3 times more frequent in one homoeologous chromosome than in the other (Woodhouse, et al. 2010). This observation is particularly relevant to maize microRNAs genes with average length distributions at the 5′ regions of their primary microRNAs (pri-miRNAs) in the order of 100 to 300 nt (Zhang, et al. 2009). Although we detected chromosome breaks of the MIR169 neighboring gene COL14 on the maize homoeologous chr1-chr4 pair (FIG. 4) and the bHLH gene on maize homeologous chr2-chr7 pair (FIG. 7), retention of the bHLH gene copy on both homoeologous regions from chr1 and chr4 was observed (FIG. 4). It has been observed that transcription factors are preferentially retained after whole genome duplication (WGD) (Murat, et al. 2010; Xu and Messing 2008), with a recent study showing that from 2,943 sorghum-maize syntenic shared genes, 43% of them were retained as homoeologous pairs in maize, from which transcription factors were 4.3 times more frequently among retained genes than other functions (Woodhouse, et al. 2010).

Alignment of sorghum regions containing MIR169 gene copies on chr2 and chr7 with their respective collinear regions from Brachypodium, rice, foxtail millet and maize revealed the close linkage of MIR169 gene copies with their flanking COL14 and Bhlh genes in all five grasses examined. Furthermore, collinearity of MIR169 gene copies with either the COL14 and/or the bHLH genes extended to dicot species such as grapevine, soybean, and cassava. Previously, it was suggested that conservation of collinearity between monocot and dicot species is rather rare because of the dynamic genomic rearrangements in genomes over 130-240 mya (Jaillon, et al. 2007; Wolfe, et al. 1989). Still, conservation of synteny between rice and grapevine was also previously observed (Tang, et al. 2010). Therefore, we hypothesized that preservation of collinearity in rare cases was subject to selection even after WGD events. In support of this hypothesis, the pseudo-functionalization and higher protein divergence rate of the HLH gene in Brachypodium chr4, where the MIR169 cluster was deleted, occurred in comparison to the orthologous bHLH copy on chr3 with the MIR169e and MIR169g copies next to it. Indeed, trade-offs between sugar content and flowering time/plant height were reported in sorghum (Murray, et al. 2008). When two genes controlling linked phenotypes are in close proximity on the chromosome for selection to act on both of them, the loss of one gene releases selection pressure on the other gene, allowing it to diverge. Based on its similarity to Arabidopsis bHLH137, which was postulated as putative DELLA target gene that functions in the GA response pathway (Zentella, et al. 2007), we hypothesize that the grass homolog may function either in flowering and/or plant height, which future research will have to confirm. On the other hand, the importance of COL family proteins in the regulation of flowering time is well known (Griffiths, et al. 2003; Wenkel, et al. 2006). Collinearity between sorghum and grapevine revealed the tight association of COL14 with vvi-MIR169z and vvi-MIR169e on grapevine chr14, with the three genes contained within a 2.3 Kbp interval. Furthermore, COL14 has been recently considered a candidate gene for a flowering QTL in grapevine (Duchene, et al. 2012). With such a short physical distance between a flowering time gene and two MIR169 gene copies, it is tempting to propose that grapevine breeding for late or early flowering time could have brought different COL14 alleles together with its neighboring MIR169 genes, a process known as linkage drag. Interestingly, although we could not find extensive collinearity between sorghum and Arabidopsis thaliana as to draw a synteny graph, we did find a close association on chr5 between COL4 gene and ath-MIR169b, separated each other 61.7 Kbp (data not shown).

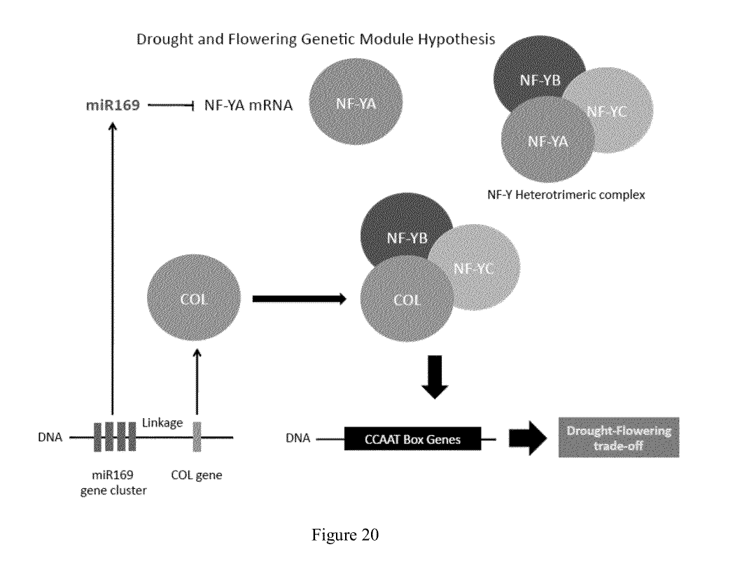

Based on these considerations, we can propose a hypothesis were the linkage of MIR169 gene copies with the neighboring COL gene could have co-evolved (FIG. 20). This hypothesis is based on the findings presented here, together with a previous report describing that CO and COL proteins can interact through their CCT domains with proteins belonging to the NF-Y (HAP) family of transcription factors (Wenkel, et al. 2006); specifically, it was described that CO together with COL 15 interacted with NF-YB and NF-YC displacing NF-YA from the ternary complex. The mRNAs encoded by the NF-YA gene family are known targets of miR169 (Li, et al. 2008). Thus, the association on the chromosome of a COL gene with a MIR169 gene or gene cluster would ensure that miR169 would reduce the expression of the NF-YA mRNA and thus its protein levels so that the COL protein can replace NF-YA in the ternary complex and drive transcription of CCAAT box genes. Furthermore, this hypothesis could provide a genetic framework where to test the previously known drought and flowering trade-offs: when plants are exposed to drought stress during the growing season they flower earlier than control plants under well watered environments (Franks, et al. 2007), with the response being genetically inherited. For this reason, we decided to term our model the “Drought and Flowering Genetic Module Hypothesis”.

We can envision a prominent role of linkage drag in breeding sorghum for enhanced biofuel traits such as high sugar content in stems and late flowering time for increased biomass. Under the MIR169-bHLH and/or MIR169-COL linkage drag model, any breeding scheme in sweet sorghum whose aim is to increase plant biomass through delayed flowering by crossing cultivars with different COL and/or bHLH alleles on either chr7 or chr2 respectively, should take into account the allelic variation at the neighboring MIR169 gene copies as they may affect sugar content in stems as well as drought tolerance. The same can be said in breeding sorghum for grain production where the norm is to increase germplasm diversity among grain sorghums through the introduction of dwarf and early flowering genes from a donor line into exotic tall and late flowering lines with African origins (Brown, et al. 2008).

Based on our results from comparative genomics analysis, we envision that any conservation in collinearity between closely associated genes (in this particular study between an microRNA and a protein-coding gene) controlling related phenotypes that is conserved among several plant species might be subject to linkage drag through breeding, opening a new area of research in genomics assisted breeding. In support of this notion, the early development of conserved ortholog set markers (referred as COS markers) among different plant species (Fulton, et al. 2002) highlighted the existence of a set of genes with synteny conservation because of the early radiation of dicotyledoneous plants that can be used in mapping through comparative genomics. In addition, conservation in linkage between candidate genes for seed glucosinolate content and SSR markers between Arabidopsis and oilseed rape (Brassica napus ssp. napus) were used in marker-assisted selection in breeding oilseed rape for total glucosinolate content (Hasan, et al. 2008).

### REFERENCES

Allen E, et al. 2004. Evolution of microRNA genes by inverted duplication of target gene sequences in Arabidopsis thaliana. Nature Genetics 36: 1282-1290. doi: 10.1038/ng1478
Axtell M J, Bowman J L 2008. Evolution of plant microRNAs and their targets. Trends in plant science 13: 343-349. doi: 10.1016/j.tplants.2008.03.009
Bennetzen J L, et al. 2012. Reference genome sequence of the model plant Setaria. Nature biotechnology. doi: 10.1038/nbt.2196
Brown P J, Rooney W L, Franks C, Kresovich S 2008. Efficient mapping of plant height quantitative trait loci in a sorghum association population with introgressed dwarfing genes. Genetics 180: 629-637. doi: 10.1534/genetics.108.092239
Calvino M, Bruggmann R, Messing J 2011. Characterization of the small RNA component of the transcriptome from grain and sweet sorghum stems. BMC genomics 12: 356. doi: 10.1186/1471-2164-12-356
Calvino M, Bruggmann R, Messing J 2008. Screen of Genes Linked to High-Sugar Content in Stems by Comparative Genomics. Rice 1: 166-176. doi: 10.1007/s12284-008-9012-9
Calvino M, Messing J 2011. Sweet sorghum as a model system for bioenergy crops. Current opinion in biotechnology 23: 1-7. doi: 10.1016/j.copbio.2011.12.002
Calvino M, Miclaus M, Bruggmann R, Messing J 2009. Molecular Markers for Sweet Sorghum Based on Microarray Expression Data. Rice 2: 129-142. doi: 10.1007/s12284-009-9029-8
Dai X, Zhao P X 2011. psRNATarget: a plant small RNA target analysis server. Nucleic Acids Research 39: W155-159. doi: 10.1093/nar/gkr319
Duchene E, Butterlin G, Dumas V, Merdinoglu D 2012. Towards the adaptation of grapevine varieties to climate change: QTLs and candidate genes for developmental stages. Theoretical and Applied Genetics 124: 623-635. doi: 10.1007/s00122-011-1734-1
Fahlgren N, et al. 2007. High-throughput sequencing of Arabidopsis microRNAs: evidence for frequent birth and death of MIRNA genes. PloS one 2: e219-. doi: 10.1371/journal.pone.0000219
Fenselau de Felippes F, Schneeberger K, Dezulian T, Huson D H, Weigel D 2008. Evolution of Arabidopsis thaliana microRNAs from random sequences. RNA 14: 2455-2459. doi: 10.1261/rna.1149408
Fernandez M G S, Becraft P W, Yin Y, Luebberstedt T 2009. From dwarves to giants? Plant height manipulation for biomass yield. Trends in plant science 14: 454-461. doi: 10.1016/j.tplants.2009.06.005
Franks S J, Sim S, Weis A E 2007. Rapid evolution of flowering time by an annual plant in response to a climate fluctuation. Proceedings of the National Academy of Sciences of the United States of America 104: 1278-1282. doi: 10.1073/pnas.0608379104
Fulton T, Van der Hoeven R, Eannetta N, Tanksley S 2002. Identification, analysis, and utilization of conserved ortholog set markers for comparative genomics in higher plants. The Plant cell 14: 1457-1467. doi: 10.1105/tpc.010479
Griffiths S, Dunford R P, Coupland G, Laurie D A 2003. The Evolution of CONSTANS-Like Gene Families in Barley, Rice, and Arabidopsis. Plant Physiology 131: 1855-1867. doi: 10.1104/pp. 102.016188
Hasan M, et al. 2008. Association of gene-linked SSR markers to seed glucosinolate content in oilseed rape (Brassica napus ssp. napus). TAG. Theoretical and applied genetics. Theoretische and angewandte Genetik 116: 1035-1049. doi: 10.1007/s00122-008-0733-3
Initiative I B 2010. Genome sequencing and analysis of the model grass Brachypodium distachyon. Nature 463: 763-768. doi: 10.1038/nature08747
Jaillon O, et al. 2007. The grapevine genome sequence suggests ancestral hexaploidization in major angiosperm phyla. Nature 449: 463-U465. doi: 10.1038/nature06148
Jiang D, et al. 2006. Duplication and expression analysis of multicopy miRNA gene family members in Arabidopsis and rice. Cell Research 16: 507-518. doi: 10.1038/sj.cr.7310062
Li W X, et al. 2008. The Arabidopsis NFYA5 Transcription Factor Is Regulated Transcriptionally and Posttranscriptionally to Promote Drought Resistance. THE PLANT CELL ONLINE 20: 2238-2251. doi: 10.1105/tpc.108.059444
Ma Z, Coruh C, Axtell M J 2010. Arabidopsis lyrata small RNAs: transient MIRNA and small interfering RNA loci within the Arabidopsis genus. The Plant cell 22: 1090-1103. doi: 10.1105/tpc.110.073882
Maher C, Stein L, Ware D 2006. Evolution of Arabidopsis microRNA families through duplication events. Genome Research 16: 510-519. doi: 10.1101/gr.4680506
Meng Y, Shao C, Gou L, Jin Y, Chen M 2011. Construction of microRNA- and microRNA*-mediated regulatory networks in plants. RNA Biology 8: 1124-1148.
Messing J, et al. 2004. Sequence composition and genome organization of maize. Proceedings of the National Academy of Sciences of the United States of America 101: 14349-14354.
Meyers B C, et al. 2008. Criteria for annotation of plant MicroRNAs. The Plant cell 20: 3186-3190. doi: 10.1105/tpc.108.064311
Murat F, et al. 2010. Ancestral grass karyotype reconstruction unravels new mechanisms of genome shuffling as a source of plant evolution. Genome Res 20: 1545-1557. doi: gr.109744.110 [pii]
   10.1101/gr.109744.110
Murray S C, et al. 2008. Genetic Improvement of Sorghum as a Biofuel Feedstock: I. QTL for Stem Sugar and Grain Nonstructural Carbohydrates. Crop science 48: 2165. doi: 10.2135/cropsci2008.01.0016
Nozawa M, Miura S, Nei M 2012. Origins and evolution of microRNA genes in plant species. Genome biology and evolution 4: 230-239. doi: 10.1093/gbe/evs002
Paterson A H, et al. 2009. The Sorghum bicolor genome and the diversification of grasses. Nature 457: 551-556. doi: 10.1038/nature07723
Piriyapongsa J, Jordan I K 2008. Dual coding of siRNAs and miRNAs by plant transposable elements. RNA 14: 814-821. doi: 10.1261/rna.916708
Sun J, Zhou M, Mao Z, Li C 2012. Characterization and Evolution of microRNA Genes Derived from Repetitive Elements and Duplication Events in Plants. PloS one 7: e34092. doi: 10.1371/journal.pone.0034092
Swigonova Z, et al. 2004. Close split of sorghum and maize genome progenitors. Genome research 14: 1916-1923.
Tamura K, et al. 2011. MEGA5: molecular evolutionary genetics analysis using maximum likelihood, evolutionary distance, and maximum parsimony methods. Molecular biology and evolution 28: 2731-2739. doi: 10.1093/molbev/msrl21
Tang H, Bowers J E, Wang X, Paterson A H 2010. Angiosperm genome comparisons reveal early polyploidy in the monocot lineage. PNAS 107: 472-477. doi: 10.1073/pnas.0908007107
Toledo-Ortiz G 2003. The Arabidopsis Basic/Helix-Loop-Helix Transcription Factor Family. The Plant cell 15: 1749-1770. doi: 10.1105/tpc.013839
Valverde F 2011. CONSTANS and the evolutionary origin of photoperiodic timing of flowering. Journal of Experimental Botany 62: 2453-2463. doi: 10.1093/jxb/erq449
Wenkel S, et al. 2006. CONSTANS and the CCAAT Box Binding Complex Share a Functionally Important Domain and Interact to Regulate Flowering of Arabidopsis. The Plant cell 18: 2971-2984. doi: 10.1105/tpc.106.043299
Wolfe K H, Gouy M, Yang Y W, Sharp P M, Li W H 1989. Date of the monocot-dicot divergence estimated from chloroplast DNA sequence data. Proceedings of the National Academy of Sciences of the United States of America 86: 6201-6205. doi: 10.1073/pnas.86.16.6201
Woodhouse M R, et al. 2010. Following Tetraploidy in Maize, a Short Deletion Mechanism Removed Genes Preferentially from One of the Two Homeologs. PLoS biology 8: e1000409. doi: 10.1371/journal.pbio.1000409.t002
Xu J-H, Messing J 2008. Diverged Copies of the Seed Regulatory Opaque-2 Gene by a Segmental Duplication in the Progenitor Genome of Rice, Sorghum, and Maize. Mol Plant % R 10.1093/mp/ssn038 1: 760-769.
Xue L-J, Zhang J-J, Xue H-W 2009. Characterization and expression profiles of miRNAs in rice seeds. Nucleic Acids Research 37: 916-930. doi: 10.1093/nar/gkn998
Yang J S, et al. 2011. Widespread regulatory activity of vertebrate microRNA* species. RNA (New York, N.Y.) 17: 312-326. doi: 10.1261/rna.2537911
Zentella R, et al. 2007. Global Analysis of DELLA Direct Targets in Early Gibberellin Signaling in Arabidopsis. The Plant cell 19: 3037-3057. doi: 10.1105/tpc.107.054999
Zhang G, et al. 2012. Genome sequence of foxtail millet (Setaria italica) provides insights into grass evolution and biofuel potential. Nature biotechnology. doi: 10.1038/nbt.2195
Zhang L, et al. 2009. A genome-wide characterization of microRNA genes in maize. PLoS genetics 5: e1000716-. doi: 10.1371/journal.pgen.1000716

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A composition comprising at least one miRNA provided in the figures in a biologically compatible carrier, for modulating expression of a plant target gene, said gene encoding a protein which regulates a biological parameter selected from the group consisting of flowering, sugar metabolism, stress response and drought resistance.

2. The composition of claim 1, wherein said at least one miRNA is cloned into an expression vector.

3. The composition of claim 1, wherein said miRNA is miR169 and said biological parameter is sugar metabolism.

4. The composition of claim 4, wherein said miR169 hybridizes to at least one gene target in FIG. 15.

5. A method for modulating a biological parameter selected from the group consisting of flowering, sugar metabolism, and stress response in a plant or plant cell comprising contacting said plant or plant cell with an effective amount of the composition as claimed in claim 1 or claim 2.

6. The method of claim 5, wherein said miRNA is effective to modulate flowering time in said plant.

7. The method of claim 5, wherein said miRNA is effective to modulate sugar metabolism in said plant.

8. A plant comprising the composition of claim 1 or 2.
